(19) United States
(12) Patent Application Publication
(10) Pub. No.: US 2013/0203734 A1
(43) Pub. Date: Aug. 8, 2013

(54) ANTI-INFLAMMATORY AGENTS

(71) Applicants: David John Grainger, Cambridge (GB); David John Fox, Coventry (GB)

(72) Inventors: David John Grainger, Cambridge (GB); David John Fox, Coventry (GB)

(21) Appl. No.: 13/702,872

(22) PCT Filed: Jun. 8, 2011
(86) PCT No.: PCT/GB11/00862

§ 371 Date: Mar. 20, 2013

(30) Foreign Application Priority Data
Jun. 8, 2010 (GB) 1009603.0

(51) Int. Cl. C07D 223/12; C07D 211/74

(52) U.S. Cl. CPC C07D 223/12; C07D 211/74
USPC 514/212.03; 514/327; 540/527; 546/216

(57) ABSTRACT

Disclosed herein are methods of preventing or treating inflammatory diseases using sulfonamide analogs of 3-aminolactam compounds, each with aromatic “tail groups”. Compounds as defined by formulae (I) and (I′), and the medical uses of the compounds, are described herein.

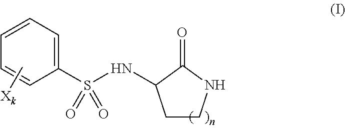

The invention relates to aryl substituted 3-aminolactam derivatives and their use in preventing or treating inflammatory diseases.

Inflammation is an important component of physiological host defence. Increasingly, however, it is clear that temporally or spatially inappropriate inflammatory responses play a part in a wide range of diseases, including those with an obvious leukocyte component (such as autoimmune diseases, asthma or atherosclerosis) but also in diseases that have not traditionally been considered to involve leukocytes (such as osteoporosis or Alzheimer's disease).

The chemokines are a large family of signalling molecules with homology to interleukin-8 which have been implicated in regulating leukocyte trafficking both in physiological and pathological conditions. With more than fifty ligands and twenty receptors involved in chemokine signalling, the system has the requisite information density to address leukocytes through the complex immune regulatory processes from the bone marrow, to the periphery, then back through secondary lymphoid organs. However, this complexity of the chemokine system has at first hindered pharmacological approaches to modulating inflammatory responses through chemokine receptor blockade. It has proved difficult to determine which chemokine receptor(s) should be inhibited to produce therapeutic benefit in a given inflammatory disease.

More recently, a family of agents which block signalling by a wide range of chemokines simultaneously has been described (see Reckless et al., Biochem J. (1999) 340: 803-811). The first such agent, a peptide termed “Peptide 3”, was found to inhibit leukocyte migration induced by 5 different chemokines, while leaving migration in response to other chemoattractants (such as fMLP or TGF-beta) unaltered. This peptide, and its analogs such as NR58-3.14.3 (i.e. c(DCys-DGln-DIle-DTrp-DLys-DGIn-DLys-DPro-DAsp-DLeu-DCys)-NH₂ [SEQ ID NO: 1]), are collectively termed “Broad Spectrum Chemokine Inhibitors” (BSCIs). Grainger et al. (2003, Biochem. Pharm. 65: 1027-1034) have subsequently shown BSCIs to have potentially useful anti-inflammatory activity in a range of animal models of diseases. Interestingly, simultaneous blockade of multiple chemokines is not apparently associated with acute or chronic toxicity, suggesting this approach may be a useful strategy for developing new anti-inflammatory medications with similar benefits to steroids but with reduced side-effects. This beneficial risk:benefit profile most likely results from the unexpected mechanism of action of these compounds (see International Patent Appl. No. PCT/GB2010/000354 in the name of Cambridge Enterprise Limited filed 28 Feb. 2010, and International Patent Appl. No. PCT/GB2010/000342 in the name of Cambridge Enterprise Limited filed 26 Feb. 2010).

However, peptides and peptoid derivatives such as NR58-3.14.3, may not be optimal for use in vivo. They are quite expensive to synthesise and have relatively unfavourable pharmacokinetic and pharmacodynamic properties. For example, NR58-3.14.3 is not orally bioavailable and is cleared from blood plasma with a half-life period of less than 30 minutes after intravenous injection.

Two parallel strategies have been adopted to identify novel preparations that retain the anti-inflammatory properties of peptide 3 and NR58-3.14.3, but have improved characteristics for use as pharmaceuticals. Firstly, a series of peptide analogs have been developed, some of which have longer plasma half-lives than NR58-3.14.3 and which are considerably cheaper to synthesise (see for example WO2009/017620). Secondly, a detailed structure: activity analysis of the peptides has been carried out to identify the key pharmacophores and design small non-peptidic structures which retain the beneficial properties of the original peptide.

This second approach yielded several structurally distinct series of compounds that retained the anti-inflammatory properties of the peptides, including 16-amino and 16-aminoalkyl derivatives of the alkaloid yohimbine, as well as a range of N-substituted 3-aminoglutarimides, identified from a small combinatorial library (see Fox et al., 2002, J Med Chem 45: 360-370; WO 99/12968 and WO 00/42071). All of these compounds are broad-spectrum chemokine inhibitors that retain selectivity over non-chemokine chemoattractants, and a number of them have been shown to block acute inflammation in vivo.

The most potent and selective of the above-mentioned aminoglutarimides was (S)-3-(undec-10-enoyl)-aminoglutarimide (NR58,4), which inhibited chemokine-induced migration in vitro with an ED₅₀ of 5 nM. This compound was orders of magnitude more potent than 3-aminoglutarimides with more complex acyl side chains (such as benzoyl or tert-butyloxo (Boc) groups). As a result, subsequent studies of aminoglutarimide and aminolactam BSCIs have focussed almost exclusively on compounds with simple linear and branched alkyl side chains.

However, further studies revealed that the aminoglutarimide ring was susceptible to enzymatic ring opening in serum. Consequently, for some applications (for example, where the inflammation under treatment is chronic, such as in autoimmune diseases) these compounds may not have optimal properties, and a more stable compound with similar anti-inflammatory properties may be superior.

As an approach to identifying such stable analogs, various derivatives of (S)-3-(undec-10-enoyl)-aminoglutarimide have been tested for their stability in serum. One derivative, the 6-deoxo analog (S)-3-(undec-10-enoyl)-tetrahydropyridin-2-one, is completely stable in human serum for at least 7 days at 37° C., but has considerably reduced potency compared with the parental molecule.

One such family of stable, broad spectrum chemokine inhibitors (BSCIs) are the 3-amino caprolactams, with a seven-membered monolactam ring (see, for example, WO2005/053702 and WO2006/016152). However, further useful anti-inflammatory compounds have also been generated from other 3-aminolactams with different ring size (see for example WO2006/134385). Other modifications to the lactam ring, including introduction of heteroatoms and bicyclolactam ring systems, also yield compounds with BSCI activity (see, for example, WO2006/018609 and WO2006/085096).

In general, these earlier studies have demonstrated that the BSCI activity is conferred on the molecule by the cyclic “head group” (a 3-amino lactam or imide) and defined, to an extent, the structural limitations for activity (for example, bulky substituents on the ring nitrogen are detrimental for activity, but variations in ring size have little impact). To be active as a BSCI, this “head group” must have an acyl “tail group” attached. Compounds with a 3-amino group, either free or N-alkyl substituted, bearing a positive charge at physiological pH are completely inactive as BSCIs. Previous disclosures have shown that this “tail group” can be linked to the “head group” through simple amide, sulfonamide, urea or carbamate linkers.

While the structure of the “head” group and linker are critical for BSCI activity, it has been shown that a wide variety of “tail groups” can be selected with out affecting the primary pharmacology of the compound, at least in vitro. As a result, modification of the “tail group” has been extensively used to optimise the physical and pharmaceutical properties of the compounds. Changes in the structure of the “tail group” can, for example, change the primary route of metabolism or excretion, modify the pharmacokinetics or oral bioavailability, and thus act as the primary determinant of the ADME properties of a selected compound.

Although the universe of possible “tail groups” known to retain BSCI activity for suitable aminolactam “head groups” is very large, some “tail groups” have been described as preferred. In some cases, structural features of the “tail group” have been identified which increase the potency of BSCI activity of the aminolactam compound. The most obvious such example is the introduction of 2′,2′ disubstitution, with a tetrahedral sp3 arrangement at the 2′ carbon centre in the tail group (the so-called “key carbon”), which confers a 10-fold increase in potency as a BSCI, at least in vitro, compared to a related compound lacking 2′2′-disubstitution. For example, 2′2′-dimethyldodecenanoyl-3-aminocaprolactam is 10-fold more potent as a BSCI in the MCP-1 induced THP-1 cell migration than assay than dodecanoyl-3-aminocaprolactam (as disclosed previously in WO2005/053702), or indeed any other related compound with a linear alkyl “tail group”. The increased potency for branched alkyl “tail groups” is restricted to branching at the 2′ position -3′3′-dimethyldodecanoyl-3-aminocaprolactam is no more potent than the linear alkyl analogs.

In other cases, structural features of the “tail group” have been identified which are associated with improved ADME properties. For example, the pivoyl “tail group” of 2′2′-dimethylpropanoyl-3-aminovalerolactam contributes to the unexpected, and particularly favourable, pharmaceutical properties of this molecule (as disclosed previously in WO2009/016390). In particular, the pivoyl group is resistant to metabolism, and therefore contributes to the unusually prolonged biological half-life of this compound.

In marked contrast, other possible “tail groups” have generally been less preferred. For example, compounds with a planar (sp2) carbon centre at the 2′ position (such as dodec-2′,3′-enoyl-3-aminocaprolactam) have markedly lower potency as BSCIs than compounds with corresponding saturated alkyl “tail groups”. Similarly, the data from the original library of glutarimides suggested that aromatic rings at the 2-position were also substantially less active (Fox et al., 2002, J Med Chem 45: 360-370). Taken together, these two findings have led to the reasonable assumption that aminolactams with aromatic “tail groups”, such as benzoyl or substituted benzoyl, would not be useful as BSCIs. As a result, previous disclosures of compounds with BSCI activity have all excluded such aromatic “tail groups”.

The present invention discloses a series of sulfonamide analogs of 3-aminolactam compounds, each with aromatic “tail groups”, as well as pharmaceutical compositions comprising the compounds, and medical uses of the compounds and compositions such as for the treatment of inflammatory diseases. Surprisingly, all of the compounds as set out below have substantial BSCI activity (greater than either 2′,3′-unsaturated acyl 3-aminolactams or benzoylaminoglutarimides).

In one aspect of the invention, there is provided a compound of general formula (I), or a pharmaceutically acceptable salt thereof, for use in the treatment of an inflammatory disorder:

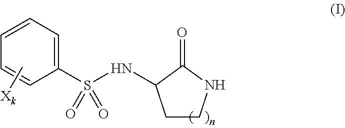

wherein

n is an integer from 1 to 4;

k is an integer from 0 to 5, representing the number of groups substituting C₂, C₃, C₄, C₅ and/or C₆ of the benzyl ring; and

X are linear or branched groups substituting the benzyl ring independently selected from any one of the group consisting of: alkyl, haloalkyl, hydroxyalkyl, hydroxy, alkoxy, amino, aminoalkyl, aminodialkyl, carboxy, and halogen.

The carbon atom at position 3 of the lactam ring is asymmetric and consequently, the compounds according to the present invention have at least two possible enantiomeric forms, that is, the “R” and “S” configurations. The present invention encompasses each of the two enantiomeric forms and all combinations of these forms, including the racemic “RS” mixtures. With a view to simplicity, when no specific configuration is shown in the structural formula, it should be understood that each of the two enantiomeric forms and their mixtures are represented.

Also provided according to the invention is a compound of formula (I′), or a pharmaceutically acceptable salt thereof, for use in the treatment of an inflammatory disorder:

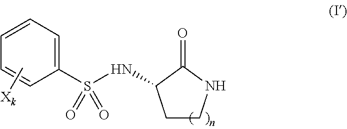

wherein n, k and X are defined as above.

Compounds (I′), having the (S)-configuration at the stereocentre, are 5-100 fold more potent as a BSCIs than the (R)-enantiomer of the same compound.

The invention additionally provides the use of a compound of general formula (I), or a pharmaceutically acceptable salt thereof, in the manufacture of a medicament for the treatment of an inflammatory disorder:

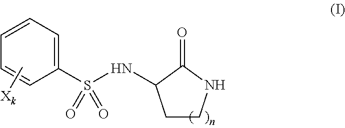

wherein

n is an integer from 1 to 4;

k is an integer from 0 to 5, representing the number of groups substituting C₂, C₃, C₄, C₅ and/or C₆ of the benzyl ring; and

X are linear or branched groups substituting the benzyl ring independently selected from any one of the group consisting of: alkyl, haloalkyl, hydroxyalkyl, hydroxy, alkoxy, amino, aminoalkyl, aminodialkyl, carboxy, and halogen.

Also provided according to the invention is the use of a compound of formula (I′), or a pharmaceutically acceptable salt thereof, in the manufacture of a medicament for the treatment of an inflammatory disorder:

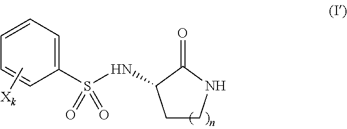

wherein n, k and X are defined as for general formula (I) above.

Certain compounds have been found to be novel per se. Thus, in another aspect of the invention, there is provided a compound of general formula (I):

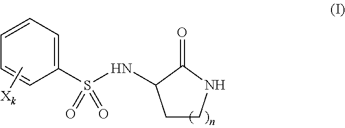

wherein n, k and X are as defined for (I) above,

with the proviso that:

when n=3, then at least one of C₂-C₆ on the benzyl ring is substituted with a group other than halogen, C₁-C₇ alkyl, or C₁-C₇ haloalkyl; and

when n=1, 2 or 3, then

C₂ or C₆ on the benzyl ring are other than hydrogen or fluorine, or

C₃ on the benzyl ring is other than hydrogen, halogen, C₁-C₆ alkyl, C₁-C₆ alkoxy, or C₁-C₆ haloalkyl, or

C₄ on the benzyl ring is other than hydrogen, halogen, C₁-C₆ alkyl, C₁-C₆ alkoxy, C₁-C₆ haloalkyl, amino, aminoalkyl or aminodialkyl, or

C₅ on the benzyl ring is other than hydrogen or halogen; and provided that the compound is neither of the group consisting of: 3-(2′-carboxybenzenesulfonylamino)-tetrahydropyridin-2-one, and (R)-3-(4′-methylbenzenesulfonylamino)-caprolactam.

For the avoidance of doubt, it is noted that according to the invention the compounds of general formula (I) do not include the compounds 3-(4′-methylbenzenesulfonylamino)-tetrahydropyridin-2-one, 3-(4′-chlorobenzenesulfonylamino)-caprolactam, 3-(4′-bromobenzenesulfonylamino)-caprolactam, (R)-3-(4′-trifluoromethylbenzenesulfonylamino)-caprolactam, 3-(4′-chlorobenzenesulfonylamino)-caprolactam, and 3 -(4′-methylbenzenesulfonylamino)-caprolactam.

Also encompassed by the invention is a compound of formula (I′):

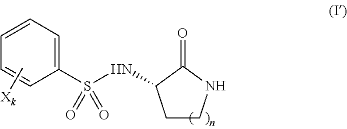

wherein n, k and X are defined as for general formula (I) above,

provided that the compound is none of the group consisting of: (S)-3-(4′-methylbenzenesulfonylamino)-tetrahydropyridin-2-one, (S)-3-(4′-methylbenzenesulfonylamino)-caprolactam, (S)-3-(4′-bromobenzenesulfonylamino)-caprolactam, and (S)-3-(4′-chlorobenzenesulfonylamino)-caprolactam.

WO2005/042489 teaches sulphonamide compounds of formula 9.0 (page 91) as intermediates for the preparation in “Scheme 3” of N-substituted benzenesulfonamides that are stated to be for use in treating cognitive disorders. Overlap with these intermediate compounds is hereby disclaimed from the present invention.

US2007/0037789 teaches fluoro substituted 2-oxo-azepan derivates as γ-secretase inhibitors. Intermediate compounds such as according to formula IV in Scheme 1 (see paragraph [0085]) are used in the synthesis of those derivates. Overlap with the intermediate compounds is hereby disclaimed from the present invention.

WO2006/005486 teaches sulphonamide derivates for the treatment of Alzheimer's disease or common cancers. Intermediate compounds such as according to formula IV (see page 12) are used in the synthesis of those derivates. Overlap with the intermediate compounds is hereby disclaimed from the present invention.

WO2007/0038669 teaches diarylamine-containing compounds and their use as modulators of c-kit receptors. Various intermediate compounds are used in the synthesis of the diarylamine-containing compounds. Any overlap of the intermediate compounds is hereby disclaimed from the present invention.

The prior art also discloses specific compounds, for example:
3-(2′-carboxybenzenesulfonylamino)-tetrahydropyridin-2-one is disclosed in Gombar et al. (1991) Quantitative Structure-Activity Relationships 10: 306-332;
3-(4′-methylbenzenesulfonylamino)-tetrahydropyridin-2-one is disclosed in Gut & Rudinger (1963) Collection of Czechoslovak Chemical Communications 28: 2953-2968;
(S)-3-(4′-methylbenzenesulfonylamino)-tetrahydropyridin-2-one is disclosed in Maguire et al. (1990) J. Organic Chem. 55: 948-955;
3-(4′-bromobenzenesulfonylamino)-caprolactam (in (S)- and (R)-forms, and with unspecified stereochemistry) and (S)-3-(4′-chlorobenzenesulfonylamino)-caprolactam are disclosed in Parker et al. (2007) Bioorganic & Medicinal Chemistry Letters 17: 5790-5795;
3-(4′-methylbenzenesulfonylamino)-caprolactam is disclosed in WO2004/033455; and
(R)-3-(4′-methylbenzenesulfonylamino)-caprolactam is disclosed in DE4117507.

However, none of the above prior art compounds have been shown to have BSCI activity, or to be useful for the treatment of inflammatory diseases. As a result, compounds disclosed in the prior art documents mentioned herein in no way teach or suggest our unexpected finding that the class of sulfonamide analogs of aryl-substituted aminolactams as defined herein have useful BSCI activity, and the prior art compounds are hereby disclaimed.

In another aspect of the invention, there is provided a pharmaceutical composition comprising, as active ingredient, a compound per se as defined above, or a pharmaceutically acceptable salt thereof, and at least one pharmaceutically acceptable excipient and/or carrier.

By pharmaceutically acceptable salt is meant in particular the addition salts of inorganic acids such as hydrochloride, hydrobromide, hydroiodide, sulphate, phosphate, diphosphate and nitrate or of organic acids such as acetate, maleate, fumarate, tartrate, succinate, citrate, lactate, methanesulphonate, p-toluenesulphonate, palmoate and stearate. Also within the scope of the present invention, when they can be used, are the salts formed from bases such as sodium or potassium hydroxide. For other examples of pharmaceutically acceptable salts, reference can be made to “Salt selection for basic drugs” (1986) Int. J. Pharm. 33: 201-217.

The pharmaceutical composition can be in the form of a solid, for example powders, granules, tablets, gelatin capsules, liposomes or suppositories. Appropriate solid supports can be, for example, calcium phosphate, magnesium stearate, talc, sugars, lactose, dextrin, starch, gelatin, cellulose, methyl cellulose, sodium carboxymethyl cellulose, polyvinylpyrrolidine and wax. Other appropriate pharmaceutically acceptable excipients and/or carriers will be known to those skilled in the art.

The pharmaceutical compositions according to the invention can also be presented in liquid form, for example, solutions, emulsions, suspensions or syrups. Appropriate liquid supports can be, for example, water, organic solvents such as glycerol or glycols, as well as their mixtures, in varying proportions, in water.

Exemplar compounds according to general formula (I) and formula (I′) for medical uses according to the invention may be selected from the group consisting of:

(S)-3-(3′-fluorobenzenesulfonylamino)-tetrahydropyridin-2-one,

(S)-3-(4′-fluorobenzenesulfonylamino)-tetrahydropyridin-2-one,

(S)-3-(2′-trifluoromethylbenzenesulfonylamino)-tetrahydropyridin-2-one,

(S)-3-(3′-trifiuoromethylbenzenesulfonylamino)-tetrahydropyridin-2-one,

(S)-3-(4′-trifluoromethylbenzenesulfonylamino)-tetrahydropyridin-2-one [also known as (S)—N-(2-oxopiperidin-3-yl)-4-(trifluoromethyl)benzenesulfonamide or (S)-3-(4′-trifluoromethylbenzenesulfonylamino)piperidin-2-one],

(S)-3-(2′,4′-difluorobenzenesulfonylamino)-tetrahydropyridin-2-one,

(S)-3-(2′,5′-difluorobenzenesulfonylamino)-tetrahydropyridin-2-one,

(S)-3-(2′,6′-difluorobenzenesulfonylamino)-tetrahydropyridin-2-one,

(S)-3-(3′,4′-difluorobenzenesulfonylamino)-tetrahydropyridin-2-one,

(S)-3-(3′,5′-difluorobenzenesulfonylamino)-tetrahydropyridin-2-one,

(S)-2-Fluoro-N-(2-oxopiperidin-3-yl)benzenesulfonamide [also known as (S)-3-(2′-fluorobenzenesulfonylamino)-tetrahydropyridin-2-one],

(S)-3-(4′-Ethylbenzenesulfonylamino)-azepan-2-one,

(S)-3-(4′-Butylbenzenesulfonylamino)-azepan-2-one,

(S)-3-(4′-tert-Butylbenzenesulfonylamino)-azepan-2-one,

(S)-3-(4′-tert-Butylbenzenesulfonylamino)-tetrahydropyridin-2-one,

(S)-3-(4′-Octylbenzenesulfonylamino)-azepan-2-one, and

(S)-3-(4′-Octylbenzenesulfonylamino)-tetrahydropyridin-2-one,

and pharmaceutically acceptable salts thereof.

Exemplar per se compounds of the invention according to general formula (I) and/or exemplar compounds according to general formula (I′) for medical uses according to the invention may be selected from the group consisting of:

(R)-3-(4′-Ethylbenzenesulfonylamino)-tetrahydropyridin-2-one,

(R)-3-(4′-tert-Butylbenzenesulfonylamino)-tetrahydropyridin-2-one,

(R)-3-(4′-Octylbenzenesulfonylamino)-tetrahydropyridin-2-one,

and pharmaceutically acceptable salts thereof.

Exemplar per se compounds of the invention according to general formula (I) and formula (I′) may be selected from the group consisting of:

(S)-3-(3′-fluorobenzenesulfonylamino)-tetrahydropyridin-2-one,

(S)-3-(4′-fluorobenzenesulfonylamino)-tetrahydropyridin-2-one,

(S)-3-(2′-trifluoromethylbenzenesulfonylamino)-tetrahydropyridin-2-one,

(S)-3-(3′-trifluoromethylbenzenesulfonylamino)-tetrahydropyridin-2-one,

(S)-3-(4′-trifluoromethylbenzenesulfonylamino)-tetrahydropyridin-2-one,

(S)-3-(2′,4′-difluorobenzenesulfonylamino)-tetrahydropyridin-2-one,

(S)-3-(2′,5′-difluorobenzenesulfonylamino)-tetrahydropyridin-2-one,

(S)-3-(2′,6′-difluorobenzenesulfonylamino)-tetrahydropyridin-2-one,

(S)-3-(3′,4′-difluorobenzenesulfonylamino)-tetrahydropyridin-2-one,

(S)-3-(3′,5′-difluorobenzenesulfonylamino)-tetrahydropyridin-2-one,

(S)-2-Fluoro-N-(2-oxopiperidin-3-yl)benzenesulfonamide,

(S)-3-(4′-Ethylbenzenesulfonylamino)-azepan-2-one,

(S)-3-(4′-Butylbenzenesulfonylamino)-azepan-2-one,

(S)-3-(4′-tert-Butylbenzenesulfonylamino)-azepan-2-one,

(S)-3-(4′-tert-Butylbenzenesulfonylamino)-tetrahydropyridin-2-one,

(S)-3-(4′-Octylbenzenesulfonylamino)-azepan-2-one, and

(S)-3 -(4′-Octylbenzenesulfonylamino)-tetrahydropyridin-2-one,

and pharmaceutically acceptable salts thereof.

An exemplar compound according to general formula (I) or (I′) for medical uses according to the invention is (S)-3-(4′-methylbenzenesulfonylamino)-caprolactam, or a pharmaceutically acceptable salt thereof.

An exemplar compound per se or for medical use according to formula (I′) is (S)-3-(4′-trifluoromethylbenzenesulfonylamino)-tetrahydropyridin-2-one, or a pharmaceutically acceptable salt thereof.

According to the invention, inflammatory disorders (which term is used herein interchangeably with “inflammatory disease”) intended to be prevented or treated by the compounds of formula (I) or (I′), or pharmaceutically acceptable salts thereof or pharmaceutical compositions or medicaments containing them as active ingredients, include notably:
autoimmune diseases, for example such as multiple sclerosis, rheumatoid arthritis, lupus, irritable bowel syndrome, Crohn's disease;
vascular disorders including stroke, coronary artery diseases, myocardial infarction, unstable angina pectoris, atherosclerosis or vasculitis, e.g., Behcet's syndrome, giant cell arteritis, polymyalgia rheumatica, Wegener's granulomatosis, Churg-Strauss syndrome vasculitis, Henoch-Schönlein purpura and Kawasaki disease;
asthma, and related respiratory disorders such as allergic rhinitis and COPD;
organ transplant rejection and/or delayed graft or organ function, e.g. in renal transplant patients;
psoriasis;
skin wounds and other fibrotic disorders including hypertrophic scarring (keloid formation), adhesion formations following general or gynaecological surgery, lung fibrosis, liver fibrosis (including alcoholic liver disease) or kidney fibrosis, whether idiopathic or as a consequence of an underlying disease such as diabetes (diabetic nephropathy); or
allergies.

The inflammatory disorder may be selected from the group consisting of autoimmune diseases, asthma, rheumatoid arthritis, a disorder characterised by an elevated TNF-α level, psoriasis, allergies, multiple sclerosis, fibrosis (including diabetic nephropathy), and formation of adhesions.

The above clinical indications fall under the general definition of inflammatory disorders or disorders characterized by elevated TNFα levels.

In one aspect of the invention, merely in order to circumvent any potentially conflicting prior art (for example as noted above), the term inflammatory disorder may exclude cognitive disorders such as Alzheimer's disease and/or memory loss.

Compounds of formula (I) or (I′) are particularly useful for local delivery, and also for the preparation of medicaments for local delivery, including creams and ointments for topical delivery, powders, aerosols or emulsions for inhaled delivery, and solutions or emulsions for injection. Pharmaceutical compositions containing one or more excipients suitable for such local delivery are therefore envisaged, and subsequently claimed.

Also provided according to the invention is a method of treatment, amelioration or prophylaxis of the symptoms of an inflammatory disease (including an adverse inflammatory reaction to any agent) by the administration to a patient of an anti-inflammatory amount of a compound, pharmaceutical composition or medicament as defined herein.

Administration of a compound, composition or medicament according to the invention can be carried out by topical, oral, parenteral route, by intramuscular injection, etc.

The administration dose envisaged for a compound, composition or medicament according to the invention is comprised between 0.1 mg and 10 g depending on the formulation and route of administration used.

The invention further encompasses a library consisting of elements all of which have structures according to the formula (I) or (I′), and hence which all have anti-inflammatory activity, useful for screening compounds for novel or improved properties in a particular assay of anti-inflammatory activity.

The invention includes compounds, compositions and uses thereof as defined, wherein the compound is in hydrated or solvated form. Unless specified otherwise, compounds of the invention include tautomers, resolved enantiomers, resolved diastereomers, racemic mixtures, solvates, metabolites, salts and prodrugs thereof, including pharmaceutically acceptable salts and prodrugs.

In any of the compounds according to formula (I) or (I′) described above (per se and/or for medical use), n may be 2. Alternatively, n may be 3.

X may be haloalkyl, for example trifluoromethyl.

An exemplar group of compounds per se and/or for medical use according to any aspect of the invention is selected from among compounds according to formula (I) or (I′) where X is halogen or haloakyl and where k is between 1 and 3. For example, X may be fluoro or fluoroalkyl (such as trifluoromethyl) and k may be between 1 and 3.

In particular, where permissible according to the formulae herein, the benzyl ring may be monosubstituted with a group X as defined above (i.e. k =1). For example, the benzyl ring may be monosubstituted with an alkyl (such as other than para-methyl), haloalkyl (such as trifluoromethyl, for example para-trifluoromethyl [i.e. 4′-trifluoromethyl]). The benzyl ring may be monosubstituted with a halogen. The benzyl ring may be monosubstituted with ortho-carboxy (i.e. 2′-carboxy).

In one aspect, the above features for k=1 apply when n=2.

According to the invention, the compounds of general formula (I) or (I′) can be prepared using the processes described hereafter.

### DEFINITIONS

The term “about” refers to an interval around the considered value. As used in this patent application, “about X” means an interval from X minus 10% of X to X plus 10% of X, and preferably an interval from X minus 5% of X to X plus 5% of X.

The use of a numerical range in this description is intended unambiguously to include within the scope of the invention all individual integers within the range and all the combinations of upper and lower limit numbers within the broadest scope of the given range. Hence, for example, the range of 0.1mg to 10 g specified in respect of (inter alia) a dose of a compound or composition of the invention to be used is intended to include all doses between 0.1mg and 10 g and all sub-ranges of each combination of upper and lower numbers, whether exemplified explicitly or not.

As used herein, the term “comprising” is to be read as meaning or encompassing both comprising and consisting of. Consequently, where the invention relates to a “pharmaceutical composition comprising as active ingredient” a compound, this terminology is intended to cover both compositions in which other active ingredients may be present and also compositions which consist only of one active ingredient as defined.

The term “alkyl” or “alkyl group” as used herein refers to a saturated linear or branched-chain monovalent hydrocarbon radical, for example of one to twenty carbon atoms, one to twelve carbon atoms, one to six carbon atoms, one to four carbon atoms, or as otherwise specified herein. Examples of alkyl groups include, but are not limited to, methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂)₅ 2-methyl-2-butyl (—C(CHs)₂CH₂CH₃), 3-methyl-2-butyl (—CH(CH₃)CH(CH₃)₂), 3-methyl-1-butyl (—CH₂CH₂CH(CH₃)2), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CHs)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, 1-heptyl, and 1-octyl.

The term “haloalkyl” or “haloalkyl group” as used herein refers to an alkyl group (as defined above) except that one or more or all of the hydrogens of the alkyl group is 15 replaced by a halogen, which replacement can be at any site on the alkyl, including the end. Examples include, but are not limited to, CH₂F, CHF₂, CF₃, CH₂CH₂F₅ CH₂CHF₂, CH₂CF₃, CHFCF₃, CF₂CF₃, CH₂Cl, CHCl₂, CCl₃, CH₂CH₂Cl, CH₂CHCl₂, CH₂CCl₃, CHClCCl₃, and CCl₂CCl₃.

The term “halogen” (which may be abbreviated to “halo”) or “halogen group” as used herein includes fluorine (F), bromine (Br), chlorine (Cl), and iodine (I).

The term “hydroxy” or “hydroxy group” denotes the group “—OH”.

The term “hydroxyalkyl” or “hydroxyalkyl group” as used herein refers to an alkyl group (as defined above) except wherein one or more or all of the hydrogens of the alkyl group is replaced by an hydroxy group, which replacement can be at any site on the alkyl, including the end.

The term “alkoxy” or “alkoxy group” denotes an alkyl group as defined above attached via a divalent oxygen atom to the rest of the molecule. Examples include but are not limited to methoxy (—OCH₃), ethoxy, propoxy, isopropoxy, n-butoxy, sec-butoxy, tert-butoxy, pentoxy, isopentoxy, neopentoxy, hexoxy, and 3-methylpentoxy.

The term “amino” or “amino group” denotes the group “—NH₂”.

The term “aminoalkyl” or “aminoalkyl group” refers to an amino group in which one of the hydrogen atoms has been replaced by an alkyl group as defined above.

The term “aminodialkyl” or “aminodialkyl group” refers to an amino group in which both of the hydrogen atoms have been replaced by an alkyl group as defined above. The alkyl groups attached to the nitrogen atom may be different or the same.

The term “carboxy” or “carboxy group” denotes the group “—C(O)OH”.

The term “benzyl ring” (also known as a “phenyl group”) refers to a 6 carbon aryl group in compounds of general formulae (I) and (I′) shown above. For the purposes of the general formulae of the present invention, numbering to locate the carbon atoms C₂-C₆ within the benzyl ring is in a clockwise direction from C₁ which is linked to the 3-aminolactam group. However, numbering of ring carbons with respect to one or more substituent groups on the benzyl ring for specific compounds follows the IUPAC rule that the second substituent in a clockwise or counter clockwise direction is afforded the lower possible location number. Where two or more substituents are present in a specific compound, the IUPAC rule is that they are listed in alphabetical order. Location numbers on the ring are assigned according to the IUPAC rule to the substituents so that they have the lowest possible number (starting from C₁ which is linked to the 3-aminolactam group), counting in either a clockwise or counter-clockwise direction.

As would be understood by a person skilled in the art, where there are fewer than 5 groups substituting the benzyl ring in compounds of general formulae (I) and (I′), i.e., where k=0, 1 2, 3 or 4, the or each unsubstituted position is occupied by a hydrogen atom.

Unless otherwise defined, all the technical and scientific terms used here have the same meaning as that usually understood by an ordinary specialist in the field to which this invention belongs. Similarly, all the publications, patent applications, all the patents and all other references mentioned here are incorporated by way of reference (where legally permissible).

Preparation of the Compounds of General Formula (I) or (I′)

Typically, such compounds are made by coupling the “tail group” in the form of a suitably activated acid (such as an acid chloride) with the appropriate 3-aminolactam. Methods for the preparation of 3-aminolactams with 5, 6, 7 and 8 membered rings, encompassing all the compounds claimed herein, have been extensively described in the literature. For example, we have provided suitable methods for the preparation of 6-membered aminolactams from ornithine (see WO2009/016390) and 7-membered aminolactams from lysine (see WO2005/053702), as well as methods for 5- and 8-membered aminolactams (see WO2006/134385). We have described in particular detail various synthesis routes to the 6-membered aminolactam, including processes suitable for scaling up the manufacture to Kg quantities (WO2009/016390). Various other methods for the synthesis of 3-aminolactams of various ring sizes have also been described in the literature (see for example Pellegata et al., 1978, Synthesis 614-616 and Boyle et al., 1979, J Org Chem 44:4841-4847), and any suitable method for the preparation of the aminolactam “head group” may be employed in accordance with the method of the present invention.

In the second step, the 3-aminolactam product is reacted with an appropriate sulfonyl chloride, for example as previously described for 7-ring aminolactams (Fox et al., 2005, J Med Chem 48: 867-74) but using a sulfonyl chloride (RS(O₂)Cl) instead of a carboxylic acid derived acid chloride (RC(O)Cl). This reaction may be carried out, for example, in chloroform or dichloromethane. The most preferred reaction solvent is dichloromethane, and is preferably carried out in the presence of a base, for example Na₂CO₃ or triethylamine (e.g. by a method similar to that described in WO 2006/005486 or Parker et al. (2007) Bioorganic & Medicinal Chemistry Letters 17: 5790-5795). The above reaction may be carried out at ambient temperature (about 25° C.) or more generally at a temperature between 20 and 50° C. The two reactions may be carried out independently, with separation and purification of the 3-aminolactam between the reactions, or alternatively, the reactions may be performed in a single vessel without purification of the 3-aminolactam prior to its derivatisation with sulfonyl chloride.

As noted previously (see WO2009/016390) care must be exercised during the acylation reaction when preparing an enantiomerically pure compound, according to formula (I′) by acylating an enantiomerically pure 3-aminolactam. In particular, the base, such as sodium carbonate, must be added slowly continually monitoring the pH of the reaction vessel to ensure that the pH of the reaction remains below pH 9.0 throughout. Excess basicity, for example due to rapid or excessive addition of sodium carbonate, increases the racemisation of the 3-aminolactam and yields enantiomerically impure product.

The following examples are presented in order to illustrate the above procedures and should in no way be considered to limit the scope of the invention.

### FIGURES

FIG. 1 shows the chemical structure of various examples of compounds according to the invention and reference examples; and

FIG. 2 is a graph showing the results of a murine sub-lethal endotoxemia test. In the graph, column A shows data from a control group (1% CMC 10 ml/kg p.o.), column B shows data from a group treated with 10 mg/kg p.o. thalidomide, column C shows data from a group treated with 10 mg/kg p.o. (S)-3-(4′-trifluoromethylbenzenesulfonyl-amino)-tetrahydropyridin-2-one (a compound according to one embodiment of the present invention—see also Example 4 below) and column D shows data from a group treated with 1 mg/kg p.o. of the somatotaxin (S)-3-(adamantane-1-carbonyl) aminocaprolactam (see WO2006/016152). The y-axis shows levels of TNF-α in pg/ml.

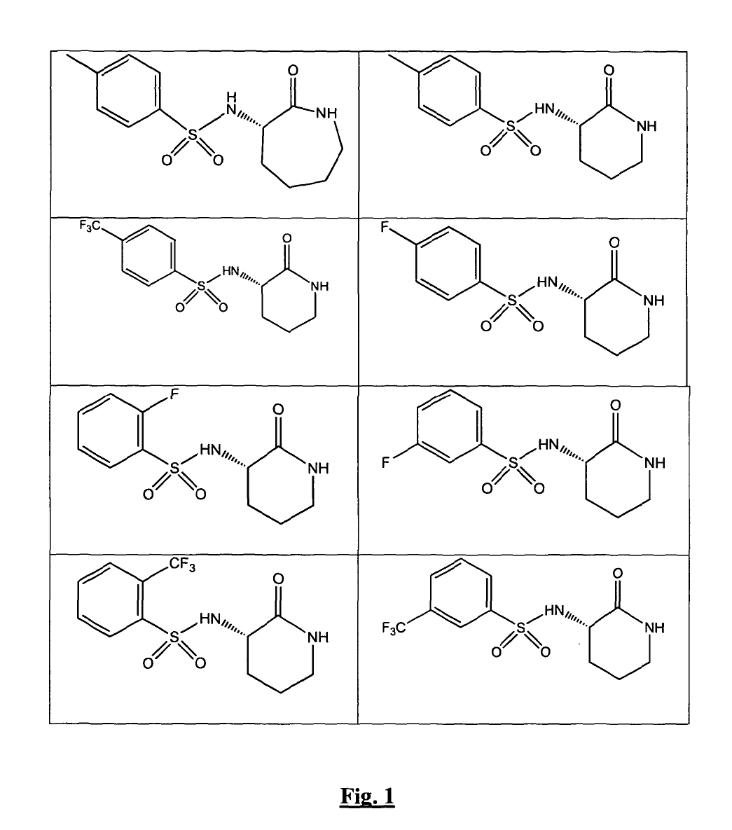

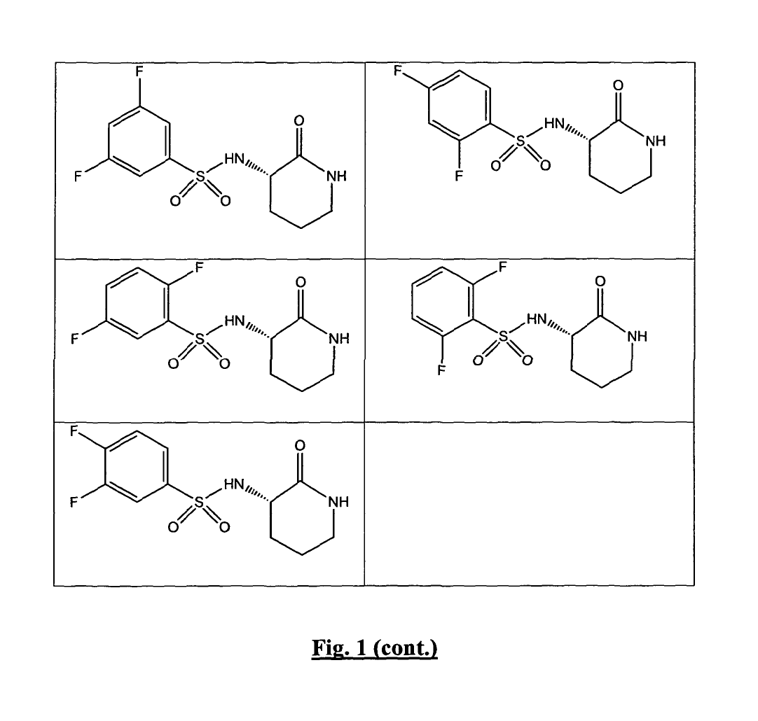

### EXAMPLES

In the following examples, ¹H-NMR and ¹³C-NMR spectra were recorded on a Bruker Avance DRX 400 MHz fourier transform machine and ¹⁹F-NMR spectra were recorded on a Bruker Avance DRX 300. Chemical shifts are given in ppm and coupling constants, J, are given in Hz to the nearest 0.5. IR spectra were recorded on an Avatar 320. HRMS data was gained via an Esquire 2000. [α]_{D} values were recorded on an optical activity AA 1000 polarimeter set at 598 nm (Sodium D line). The samples were made using spectroscopic grade MeOH.

### Reference Example 1

### 3-(4′-Methylbenzenesulfonylamino)tetrahydropyridin-2-one

3-aminotetrahydropyridin-2-one hydrochloride (10 mmol), K₂CO₃ (30 mmol) and 4-methylbenzenesulfonyl chloride (10 mmol) were reacted according to the above procedure to give the product (1.64 g, 69%):

νₘₐₓ/cm⁻¹ 3224, 1658 (secondary CONH, lactam), 1598, 1494 (aromatic ring), 1324, 1161 (SO₂—N), 814, 802 (para-disubstituted benzene).

¹H NMR: δ_{H} (400 MHz, CDCl₃) 7.77 (2H, d, J8.5, ortho-H), 7.29 (2H, d, J 8.0, meta-H), 5.79 (1H, br d, J 1.0, C₇H₇—SO₂NH), 5.56 (1H, br s, CONH—CH₂), 3.49-3.42 (1H, m, CH—CO), 3.31-3.24 (2H, m, CH₂NH), 2.53-2.45 (1H, m, lactam CH₂), 2.40 (3H, s, CH₃), 1.97-1.88 (1H, m, lactam CH₂), 1.88-1.68 (2H, m, lactam CH₂).

¹³C NMR: δ_{C} (100 MHz, CDCl₃) 172.2 (lactam C═O), 142.2 (ipso-C), 136.2 (para-C), 129.7 (aromatic CH), 127.3 (aromatic CH), 53.3 (CH—CO), 42.0 (CH₂—NH), 29.6 (lactam CH₂), 28.6 (lactam CH₂), 27.9 (lactam CH₂), 21.5 (CH₃). HRMS (+ESI) C₁₂H₁₆N₂O₃S+Na⁺: calcd 291.0774; found 291.0777.

### Reference Example 2

### 3-(4′-Methylbenzenesulfonylamino)azepan-2-one:

3-aminoazepan-2-one hydrochloride (10 mmol), K₂CO₃ (30 mmol) and 4-methylbenzenesulfonyl chloride (10 mmol) were reacted according to the above procedure to give the product (1.70 g, 67%):

νₘₐₓ/cm⁻¹ 3393, 1658 (secondary CONH, lactam), 1598, 1496 (aromatic ring), 1324, 1164 (SO₂—N), 818, 802 (para-disubstituted benzene).

¹H NMR: δ_{H} (400 MHz, CDCl₃) 7.70 (2H, d, J8.5, ortho-H), 7.25 (2H, d, J8.0, meta-H), 6.22-6.03 (2H, m, NH), 3.79 (1H, ddd, J 11.0, 5.0, 2.0, CH—CO), 3.19-3.10 (1H, m, CH₂NH), 3.08-2.98 (1H, m, CH₂NH), 2.38 (3H, s, CH₃), 2.14-2.08 (1H, m, lactam CH₂), 2.03-1.93 (1H, m, lactam CH₂), 1.82-1.71 (1H, m, lactam CH₂), 1.70-1.54 (2H, m, lactam CH₂), 1.38-1.25 (1H, m, lactam CH₂).

¹³C NMR: δ_{C} (100 MHz, CDCl₃) 174.5 (lactam C═O), 143.6 (ipso-C), 137.2 (para-C), 129.9 (aromatic CH), 127.2 (aromatic CH), 55.6 (CH—CO), 42.4 (CH₂—NH), 33.6 (lactam CH₂), 28.9 (lactam CH₂), 28.2 (lactam CH₂), 21.7 (CH₃). HRMS (+ESI) C₁₃H₁₈N₂O₃S+Na⁺: calcd 305.0930; 305.0938.

With respect to the examples below, the general procedure for the synthesis of 3-sulfonylamino-2-oxopiperidines was: potassium carbonate (3 mmol) and (S)-3-amino-2-oxopiperidine hydrochloride (1.5 mmol) were dissolved in water (5 ml) and the solution was cooled to 0° C., and a solution of substituted benzenesulfonyl chloride (1 mmol) in tetrahydrofuran (5 mL) was added. The mixture was stirred for 16 hours, and then the reaction was extracted with dichloromethane or chloroform. The combined organic layers were dried over sodium sulfate and reduced in vacuo to give a solid. This solid was redissolved in a minimum amount of dichloromethane and crystallised by addition of petroleum ether 40-60° C. The solid product was isolated by filtration and dried over potassium pentoxide.

### Example 1

### (S)-4-Fluoro-N-(2-oxopiperidin-3-yl)benzenesulfonamide

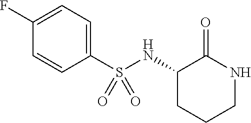 [SMILES: O=C1NCCC[C@@H]1NS(=O)(=O)c1ccc(F)cc1]

0.196 g white fine powder (48%). mp 153-156° C.; [α]²⁴_{D}+30.35 (c 0.1, MeOH); νₘₐₓ/cm⁻¹ 1656, 1650 (C═O, amide), 1493 (N—H, amide), 1329 (C—F), 1158 (—SO₂—). Anal. (C₁₁H₁₃FN₂O₃S) C, H, N: calcd C 48.52, H 4.81, N 10.29; found C 48.13, H 4.74, N 10.18. ¹H-NMR δ_{H} ¹H-NMR δ_{H}. 7.90 (2H, dd, J 9 and 5, ArH2 and ArH6), 7.17 (2H, t, J8.5, ArH3 and ArH5), 5.82 (2H, NHCH and NHCH₂), 3.48 (1H, dd, J 11 and 6, CHNH), 3.31-3.25 (2H, m, CH₂NH), 2.48-2.41 (1H, m, CH₂CH), 1.99-1.88 (2H, m, CH₂CH₂NH), 1.87-1.66 (1H, m, CH₂CH). ¹³C-NMR δ_{C} 169.74 (CHCONH), 165.1 (d, J 255, ArC4), 135.2 (d, J 3, CSO₂), 130.1 (d, J 9, ArC2/6), 116.4 (d, J 23, ArC3/5), 53.3 (CHNH), 41.9 (CH₂NH), 28.6 (CH₂CHNH), 20.8 (CH₂CH₂NH). ¹⁹F-NMR δ_{F}−105.1. HRMS (+ESI) C₁₁H₁₃FN₂O₃SNa: calcd 295.0523; found 295.0517.

### Example 2

### (S)-3-Fluoro-N-(2-oxopiperidin-3-yl)benzenesulfonamide

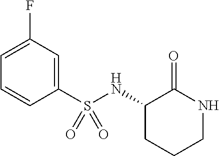 [SMILES: O=C1NCCC[C@@H]1NS(=O)(=O)c1cccc(F)c1]

0.215 g off-white fine powder (53%). mp 159-160° C.; [α]²⁴_{D}+29.80 (c 0.1, MeOH); νₘₐₓ/cm⁻¹ 1669, 1644 (C═O, amide), 1552 (N—H, amide), 1303 (C—F), 1158 (—SO₂—). Anal. (C₁₁H₁₃FN₂O₃S) C, H, N: calcd C 48.52, H 4.81, N 10.29; found C 48.10, H 4.71, N 10.05. ¹H-NMR δ_{H} 7.69 (1H, dt, J 8 and 1, ArH2), 7.60 (1H, dt, J 8 and 2.5, ArH5), 7.49 (1H, td, J 8 and 6, ArH4), 7.26 (1H, tdd, J 8, 2.5 and 1, ArH6), 5.89 (1H, s, NHCH), 5.69 (1H, s, NHCH₂), 3.52 (1H, dd, J 11 and 6, CHNH), 3.32-3.27 (2H, m, CH₂NH), 2.51-2.44 (1H, m, CH₂CH), 1.98-1.89 (2H, m, CH₂CH₂NH), 1.87-1.67 (1H, m, CH₂CH). ¹³C-NMR δ_{C} 169.1 (CHCONH), 162.4 (d, J 250, ArC3), 141.2 (d, J 7, CSO₂), 130.9 (ArC5), 123.0 (ArC6), 120.1 (d, J 21, ArC4), 114.8 (d, J 24, ArC2), 53.4 (CHNH), 41.9 (CH₂NH), 28.6 (CH₂CHNH), 20.7 (CH₂CH₂NH). ¹⁹F-NMR δ_{F}−109.4. HRMS (+ESI) C₁₁H₁₃FN₂O₃SNa: calcd 295.0523; found 295.0535.

### Example 3

### (S)-2-Fluoro-N-(2-oxopiperidin-3-yl)benzenesulfonamide

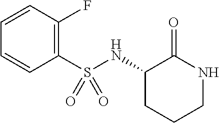 [SMILES: O=C1NCCC[C@@H]1NS(=O)(=O)c1ccccc1F]

0.197 g white powder (48%). mp 180-183° C.; [α]²⁴_{D}+35.80 (c 0.1, MeOH); νₘₐₓ/cm⁻¹ 1659, 1647 (C═O, amide), 1474 (N—H, amide), 1332 (C—F), 1157 (—SO₂—). Anal. (C₁₁H₁₃FN₂O₃S.⅙ H₂O) C, H, N: calcd C 47.99, H 4.88, N 10.18; found C 47.94, H 4.76, N 10.01. ¹H-NMR δ_{H} 7.87 (1H, td, J 7.5 and 2, ArH4), 7.56 (1H, dddd, J 8, 7, 5 and 2, ArH3), 7.21 (2H, m, J 7.5 and 1, ArH5 and ArH6), 6.05 (1H, s, NHCH), 5.84 (1H, s, NHCH₂), 3.58 (1H, dt, J 10.5 and 6, CHNH), 3.29-3.24 (2H, m, CH₂NH), 2.51-2.43 (1H, m, CH₂CH), 1.98-1.89 (2H, m, CH₂CH₂NH), 1.88-1.69 (1H, m, CH₂CH). ¹³C-NMR δ_{C} 169.6 (CHCONH), 159.2 (d, J 256, ArC2), 135.2 (d, J 9, ArC4), 130.6 (ArC5), 127.2 (d, J 14, CSO₂), 124.2 (d, J 4, ArC6), 117.2 (d, J 21, ArC3), 53.7 (CHNH), 41.9 (CH₂NH), 28.9 (CH₂CHNH), 20.9 (CH₂CH₂NH). ¹⁹F-NMR δ_{F}−108.5. HRMS (+ESI) C₁₁H₁₃FN₂O₃SNa: calcd 295.0523; found 295.0516.

### Example 4

### (S)—N-(2-Oxopiperidin-3-yl)-4-(trifluoromethyl)-benzenesulfonamide

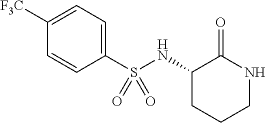 [SMILES: O=C1NCCC[C@@H]1NS(=O)(=O)c1ccc(C(F)(F)F)cc1]

0.222 g off-white fine powder (46%). mp 181-183° C.; [α]²⁴_{D}+21.15 (c 0.1, MeOH); νₘₐₓ/cm⁻¹ 1669, 1644 (C═O, amide), 1552 (N—H, amide), 1303 (C—F), 1158 (—SO₂—). Anal. (C₁₂H₁₃F₃N₂O₃S) C, H, N: calcd C 44.72, H 4.07, N 8.69; found C 44.39, H 3.98, N 8.54. ¹H-NMR δ_{H}. 8.03 (2H, d, J 8, ArH2 and ArH6), 7.76 (2H, d, J 8, ArH3 and ArH5), 5.99 (1H, s, NHCH), 5.85 (1H, s, NHCH₂), 3.53 (1H, dd, J 11 and 6, CHNH), 3.31-3.26 (2H, m, CH₂NH), 2.49-2.42 (1H, m, CH₂CH), 1.99-1.91 (2H, m, CH₂CH₂NH), 1.89-1.67 (1H, m, CH₂CH). ¹³C-NMR δ_{C} 169.5 (CHCONH), 142.9 (CSO₂), 134.5 (q, J 32, ArC4), 127.9 (ArC2/6), 126.3 (q, J 4, ArC3/5), 123.3 (q, J 270, CF₃), 53.4 (CHNH), 41.9 (CH₂NH), 28. 7 (CH₂CHNH), 20.8 (CH₂CH₂NH). ¹⁹F-NMR δ_{F}−63.1. HRMS (+ESI) C₁₂H₁₃F₃N₂O₃SNa: calcd 345.0491; found 345.0478.

### Example 5

### (S)—N-(2-Oxopiperidin-3-yl)-3-(trifluoromethyl)benzenesulfonamide

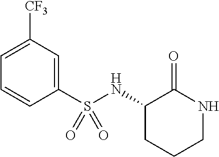 [SMILES: O=C1NCCC[C@@H]1NS(=O)(=O)c1cccc(C(F)(F)F)c1]

0.156 g off-white needle crystals (32%). mp 158-160° C.; [α]²⁴_{D}+22.90 (c 0.1, MeOH); νₘₐₓ/cm⁻¹ 1632, 1614 (C═O, amide), 1496 (N—H, amide), 1362 (C—F), 1138 (—SO₂—). Anal. (C₁₁H₁₃F₃N₂O₃S) C, H, N: calcd C 44.72, H 4.07, N 8.69; found C 44.56, H 3.93, N 8.63. ¹H-NMR δ_{H} ¹H-NMR δ.8.17 (1H, s, ArH2), 8.09 (1H, d, J 8, ArH6), 7.82 (1H, d, J 8, ArH4), 7.66 (1H, t, J 7.5, ArH5), 5.97 (1H, s, NHCH), 5.83 (1H, s, NHCH₂), 3.55 (1H, dd, J 10.5 and 6, CHNH), 3.32-3.26 (2H, m, CH₂NH), 2.49-2.41 (1H, m, CH₂CH), 1.98-1.89 (2H, m, CH₂CH₂NH), 1.87-1.67 (1H, m, CH₂CH). ¹³C-NMR δ_{C} 169.6 (CHCONH), 140.7 (CSO₂), 131.7 (q, J34, ArC3), 130.5 (ArC5), 129.9 (ArC6), 129.4 (q, J 3, ArC2), 124.5 (q, J 3, ArC4), 123.2 (q, J 272.5, CF₃), 53.4 (CHNH), 41.9 (CH₂NH), 28.7 (CH₂CHNH), 20.8 (CH₂CH₂NH). ¹⁹F-NMR 8_{F}−62.7. HRMS (+ESI) C₁₂H₁₃F₃N₂O₃SNa: calcd 345.0491; found 345.0480.

### Example 6

### (S)—N-(2-oxopiperidin-3-yl)-2-(trifluoromethyl)benzenesulfonamide

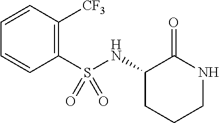 [SMILES: O=C1NCCC[C@@H]1NS(=O)(=O)c1ccccc1C(F)(F)F]

0.191 g off-white fine powder (40%). mp 161-163° C.; [α]²⁴_{D}+36.70 (c 0.1, MeOH); νₘₐₓ/cm⁻¹ 1669, 1644 (C═O, amide), 1552 (N—H, amide), 1303 (C—F), 1158 (—SO₂—). Anal. (C₁₁H₁₃F₃N₂O₃S) C, H, N: calcd C 44.72, H 4.07, N 8.69; found C 44.51, H 3.89, N 8.62. ¹H-NMR δ_{H}. 7.74 (1H, td, J 9 and 3.5, ArH6), 7.42 (1H, dt, J 9and 4, ArH3), 7.22-7.15 (2H, m, ArH4 and ArH5), 6.78 (1H, d, J 5, NHCH), 6.08 (1H, s, NHCH₂), 4.42 (1H, dt, J 11 and 6, CHNH), 3.35 (2H, td, J 6 and 2, CH₂NH), 2.73 (1H, dq, J 13 and 6, CH₂CH), 1.99-1.91 (2H, m, CH₂CH₂NH), 1.59 (1H, dq, J 12 and 8, CH₂CH). ¹³C-NMR δ_{C} 169.9 (CHCONH), 138.1 (CSO₂), 132.8 (ArCS), 132.2 (ArC6), 131.1 (ArC4), 128.7 (q, J 6, ArC3), 128.1 (q, J 34, ArC2), 122.9 (q, J 273, CF₃), 53.7 (CHNH), 41.9 (CH₂NH), 28.9 (CH₂CHNH), 20.9 (CH₂CH₂NH). ¹⁹F-NMR 8_{F} -57.9. HRMS (+ESI) C₁₂H₁₃F₃N₂O₃SNa: calcd 345.0491; found 345.0502.

### Example 7

### (S)-2,4-difluoro-N-(2-oxopiperidin-3-yl)benzenesulfonamide

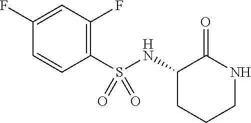 [SMILES: O=C1NCCC[C@@H]1NS(=O)(=O)c1ccc(F)cc1F]

0.193 g off-white fine powder (44%). mp 162-163° C.; [α]²⁴_{D}+31.55 (c 0.1, MeOH); νₘₐₓ/cm⁻¹ 1679, 1655 (C═O, amide), 1475 (N—H, amide), 1342 (C—F), 1160 (—SO₂—). Anal. (C₁₁H₁₂F₂N₂O₃S) C, H, N: calcd C 45.51, H 4.17, N 9.65; found C 45.37, H 4.14, N 9.38. ¹H-NMR δ_{H}. 7.94 (1H, td, J 8.5 and 6, ArH3), 6.9 (1H, m, ArH5 and ArH6), 6.09 (1H, s, NHCH), 6.03 (1H, s, NHCH₂), 3.65 (1H, dt, J 11 and 5.5, CHNH), 3.35-3.29 (2H, m, CH₂NH), 2.52-2.45 (1H, m, CH₂CH), 2.03-1.95 (2H, m, CH₂CH₂NH), 1.92-1.75 (1H, m, CH₂CH). ¹³C-NMR δ_{C} 169.7 (CHCONH), 165.9 (dd, J 260 and 11.5, ArC2), 160.1 (dd, J 258 and 13, ArC4), 131.9 (d, J 10.5, ArC6), 123.9 (dd, J 12.5 and 3.5, CSO₂), 111.6 (dd, J 22 and 4, ArCS), 105.8 (t, J 20, ArC3), 53.6 (CHNH), 41.9 (CH₂NH), 28.9 (CH₂CHNH), 20.9 (CH₂CH₂NH). ¹⁹F-NMR δ_{F}−100.5 (d, J 12), −103.3 (d, J 12). HRMS (+ESI) C₁₁H₁₂F₂N₂O₃SNa: calcd 313.0429; found 313.0440.

### Example 8

### (S)-2,5-difluoro-N-(2-oxopiperidin-3-yl)benzenesulfonamide

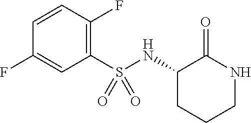 [SMILES: O=C1NCCC[C@@H]1NS(=O)(=O)c1cc(F)ccc1F]

0.234 g white fine powder (54%). mp 183-185° C.; [α]²⁴_{D}+26.30 (c 0.1, MeOH); νₘₐₓ/cm⁻¹ 1692, 1635 (C═O, amide), 1576 (N—H, amide), 1352 (C—F), 1166 (—SO₂—). Anal. (C₁₁H₁₂F₂N₂O₃S) C, H, N: calcd C 45.51, H 4.17, N 9.65; found C 45.35, H 4.11, N 9.42. ¹H-NMR δ_{H}. 7.64 (1H, ddd, J7, 5 and 3, ArH6), 7.26 (2H, tq, J 8 and 4, ArH3 and ArH4), 5.91 (2H, s, NHCH and NHCH₂), 3.69 (1H, dt, J 11.5 and 6, CHNH), 3.36-3.31 (2H, m, CH₂NH), 2.55-2.47 (1H, m, CH₂CH), 2.04-1.95 (2H, m, CH₂CH₂NH), 1.94-1.76 (1H, m, CH₂CH). ¹³C-NMR δ_{C} 171.3 (CHCONH), 169.5 (CCONH), 157.7 (dd, J248 and 2, ArC5), 155.3 (dd, J252 and 2, ArC2), 128.6 (dd, J 26 and 6, CSO₂), 121.5 (dd, J26 and 8, ArC3), 118.5 (dd, J23.5 and 8.5, ArC4), 117.0 (d, J 28, ArC6), 53.7 (CHNH), 41.9 (CH₂NH), 28.9 (CH₂CHNH), 20.9 (CH₂CH₂NH). ¹⁹F-NMR δ_{F}−114.6 (d, J 18), −115.9 (d. J 19). HRMS (+ESI) C₁₁H₁₂F₂N₂O₃SNa: calcd 313.0429; found 313.0418.

### Example 9

### (S)-2,6-difluoro-N-(2-oxopiperidin-3-yl)benzenesulfonamide

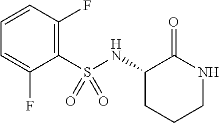 [SMILES: O=C1NCCC[C@@H]1NS(=O)(=O)c1c(F)cccc1F]

0.173 g white coarse powder (40%). mp 152-153° C.; [α]²⁴_{D}+15.95 (c 0.1, MeOH); νₘₐₓ/cm⁻¹ 1659, 1621 (C═O, amide), 1493 (N—H, amide), 1326 (C—F), 1161 (—SO₂—). Anal. (C₁₁H₁₂F₂N₂O₃S) C, H, N: calcd C 45.51, H 4.17, N 9.65; found C 45.18, H 4.10, N 9.20. ¹H-NMR δ_{H}. 7.54 (1H, qt, J 8.5 and 6, ArH4), 7.06 (2H, t, J 8.5, ArH3 and ArH5), 6.25 (1H, s, NHCH), 5.93 (1H, s, NHCH₂), 3.79 (1H, dd, J 11.5 and 6, CHNH), 3.36-3.31 (2H, m, CH₂NH), 2.60-2.55 (1H, m, CH₂CH), 2.04-1.97 (2H, m, CH₂CH₂NH), 1.87 (1H, qd, J 12 and 4, CH₂CH). ¹³C-NMR δ_{C} 169.7 (CHCONH), 159.8 (dd, J 260 and 4, ArC2 and ArC6), 134.5 (t, J 11, ArC4), 117.4 (t, J 16, CSO₂), 113.0 (dd, J 23 and 4, ArC3 and ArC5), 53.8 (CHNH), 41.8 (CH₂NH), 28.9 (CH₂CHNH), 20.9 (CH₂CH₂NH). ¹⁹F-NMR δ_{F}−107.5. HRMS (+ESI) C₁₁H₁₂F₂N₂O₃SNa: calcd 313.0429; found 313.0417.

### Example 10

### (S)-3,4-difluoro-N-(2-oxopiperidin-3-yl)benzenesulfonamide

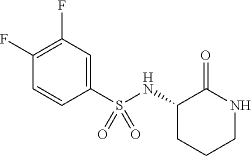 [SMILES: O=C1NCCC[C@@H]1NS(=O)(=O)c1ccc(F)c(F)c1]

0.200 g white fine powder (46%). mp 153-155° C.; [α]²⁴_{D}+25.60 (c 0.1, MeOH); νₘₐₓ/cm⁻¹ 1656, 1603 (C═O, amide), 1501 (N—H, amide), 1331 (C—F), 1160 (—SO₂—). Anal. (C₁₁H₁₂F₂N₂O₃S) C, H, N: calcd C 45.51, H 4.17, N 9.65; found C 45.39, H 4.11, N 9.49. ¹H-NMR δ_{H}. 7.79 (1H, dq, J 7 and 2, ArH5), 7.73 (1H, t, J 8.5, ArH6), 7.34 (1H, q, J 8.5, ArH2), 6.00 (2H, s, NHCH and NHCH₂), 3.59 (1H, dd, J 11 and 6, CHNH), 3.36-3.32 (2H, m, CH₂NH), 2.52-2.45 (1H, m, CH₂CH), 2.03-1.97 (2H, m, CH₂CH₂NH), 1.83 (1H, qd, J 12 and 4, CH₂CH). ¹³C-NMR δ_{C} 169.6 (CHCONH), 153.2 (dd, J 256 and 11, ArC3), 150.1 (dd, J 256 and 12, ArC4), 136.1 (t, J 4, CSO₂), 124.4 (q, J 3.5, ArC6), 118.2 (d, J 18, ArC5), 117.4 (d, J 22, ArC2), 53.4 (CHNH), 41.9 (CH₂NH), 28.7 (CH₂CHNH), 20.8 (CH₂CH₂NH). ¹⁹F-NMR δ_{F}−129.3 (d, J 18), −133.5 (d. J 19). HRMS (+ESI) C₁₁H₁₂F₂N₂O₃SNa: calcd 313.0429; found 313.0417.

### Example 11

### (S)-3,5-difluoro-N-(2-oxopiperidin-3-yl)benzenesulfonamide

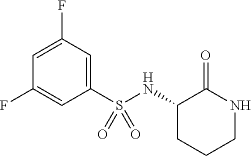 [SMILES: O=C1NCCC[C@@H]1NS(=O)(=O)c1cc(F)cc(F)c1]

0.193 g off-white fine powder (44%). mp 170-174° C.; [α]²⁴_{D}+21.10 (c 0.1, MeOH); νₘₐₓ/cm⁻¹ 1658, 1604 (C═O, amide), 1491 (N—H, amide), 1332 (C—F), 1163 (—SO₂—). Anal. (C₁₁H₁₂F₂N₂O₃S) C, H, N: calcd C 45.51, H 4.17, N 9.65; found C 45.42, H 4.12, N 9.41. ¹H-NMR δ_{H}. 7.49 (1H, ddt, J 11, 6.5 and 2.5, ArH4), 7.05 (2H, tt, J 8.5 and 2, ArH2 and ArH6), 6.09 (2H, s, NHCH and NHCH₂), 3.64 (1H, dd, J 11.5 and 6, CHNH), 3.36-3.32 (2H, m, CH₂NH), 2.52-2.45 (1H, m, CH₂CH), 2.04-1.96 (2H, m, CH₂CH₂NH), 1.84 (1H, qd, J 12 and 6, CH₂CH). ¹³C-NMR δ_{C} 169.6 (CHCONH), 162.8 (dd, J 252 and 13, ArC3 and ArCS), 142.8 (t, J 8.5, CSO₂), 110.9 (dd, J 21 and 7, ArC2 and ArC6), 108.5 (t, J 25, ArC4), 53.5 (CHNH), 41.9 (CH₂NH), 28.6 (CH₂CHNH), 20.8 (CH₂CH₂NH). ¹⁹F-NMR δ_{F}−105.4. HRMS (+ESI) C₁₁H₁₂F₂N₂O₃SNa: calcd 313.0429; found 313.0431.

### Example 12

### (S)-3-(4’-Ethylbenzenesulfonylamino)-azepan-2-one

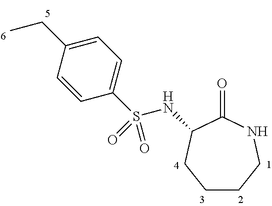 [SMILES: CCc1ccc(S(=O)(=O)N[C@H]2CCCCNC2=O)cc1]

(S)-3-amino-azepan-2-one hydrochloride (0.55 g, 3.34 mmoles) was dissolved in H₂O (20 mL) and cooled to 0° C. 4-Ethylbenzenesulfonyl chloride (5 mmoles) in dichloromethane (30 mL) was added and triethylamine (1.3 mL, 9 mmoles) and the reaction was stirred over night. The reaction was extracted with dichloromethane and washed with pH 2 buffer (3×20 mL) and reduced in vacuo. The product was purified by silica column chromatography (petroleum ether:ethyl acetate 75:25 to 0:100) to give the product as a white solid 0.17 g (19%); δ_{H} (400 MHz, CDCl₃) 7.72 (d, 2H, J8, CH—C-Et), 7.28 (d, 2H, J 8, CH—C—SO₂), 6.40 (br.t, 1H, J 6, NH—C1), 6.18 (d, 1H, J 5, NH—CH), 3.83-3.74 (m, 1H, CH—C4), 3.19-3.11 (m, 1H, H1), 3.03 (ddd, 1H, J16, 11.5, 5.5, H1), 2.08 (q, 2H, J 8, H5), 2.15-2.09 (m, 1H, H4), 2.0-1.95 (m, 1H, H2), 1.80-1.77 (m, 1H, H3), 1.66-1.52 (m, 2H, H3 & H4), 1.37-1.26 (m, 1H, H2) and 1.23 (t, 3H, J 8, H6); δ_{C} (100 MHz, CDCl₃) 174.4 (C═O), 149.5 (C-Et), 137.2 (C—SO₂), 126.2 (CH phenyl), 55.4 (CH—NH), 42.2 (C1), 33.4 (C4), 28.8 (C5), 28.6 (C2), 28.0 (C3) and 15.1 (C6); ESI m/z 100%, 319.1 (MNa⁺) and 58%, 614.6 (M₂Na⁺); HR ESI m/z (C₁₁₄H₂₀N₂O₃SNa⁺ requires 319.1087) found 319.1085; [α]²⁴_{D} (c=0.235, CHCl₃) +128.79.

### Example 13

### (R)-3-(4′-Ethylbenzenesulfonylamino)-tetrahydropyridin-2-one

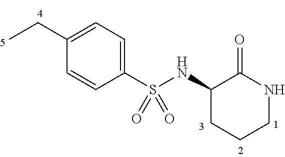 [SMILES: CCc1ccc(S(=O)(=O)N[C@@H]2CCCNC2=O)cc1]

(R)-3-amino-γ-lactam (4 mmoles) was dissolved in H₂O (20 mL) and cooled to 0° C. 4-Ethylbenzenesulfonyl chloride (4 mmoles) in dichloromethane (25 mL) was added and triethylamine (1.7 mL, 12 mmoles) and the reaction was stirred over night. The reaction was extracted with dichloromethane and washed with pH 2 buffer (3×20 mL) and reduced in vacuo. The product was purified by silica column chromatography (petroleum ether:ethyl acetate:methanol 75:25:0 to 0:90:10) to give the product as a white solid 0.25 g (21%); δ_{C} (100 MHz, CDCl₃) 170.0 (C═O), 149.7 (C-Et), 136.2 (C—SO₂), 129.1, 128.6, 127.5 (CH phenyl), 55.3 (CH—NH), 41.9 (C1), 28.8 (C3), 28.5 (C4), 20.8 (C2) and 15.1 (C5); ESI m/z 100%, 305.1 (MNa⁺) and 56%, 586.7 (M₂Na⁺); HR ESI m/z (C₁₃H₁₈N₂O₃SH⁺ requires 283.1111) found 283.1114;

### Example 14

### (S)-3-(4′-Butylbenzenesulfonylamino)-azepan-2-one

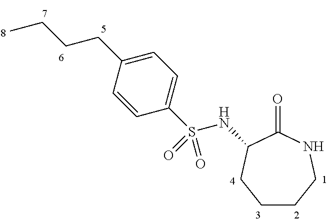 [SMILES: CCCCc1ccc(S(=O)(=O)N[C@H]2CCCCNC2=O)cc1]

(S)-3-amino-azepan-2-one hydrochloride (1.15 g, 7 mmoles) was dissolved in H₂O (20 mL) and cooled to 0° C. 4-Butylbenzenesulfonyl chloride (7 mmoles) in dichloromethane (30 mL) was added and triethylamine (2.95 mL, 21 mmoles) and the reaction was stirred over night. The reaction was extracted with dichloromethane and washed with pH 2 buffer (3×20 mL) and reduced in vacuo. The product was purified by silica column chromatography (petroleum ether:ethyl acetate 75:25 to 0:100) to give the product as a white solid. δ_{H} (400 MHz, CDCl₃) 7.76 (d, 2H, J 8, CH—CBu), 7.31 (d, 2H, J 7.5, CH—C—SO₂), 6.76 (br.t, 1H, J 6, NH—C1), 6.28 (d, 1H, J 5, NH—CH), 3.85 (ddd, 1H, J 11, 5, 2, CH—C4), 3.24-3.16 (m, 1H, H1), 3.06 (ddd, 1H, J 15, 12, 5, H1), 2.68 (t, 2H, J 8, H5), 2.18-2.11 (m, 1H, H4), 2.03-1.93 (m, 1H, H3), 1.84-1.73 (m, 1H, H2), 1.69-1.55 (m, 4H, H3, H4 & H6), 1.38 (sextet, 2H, J 7.5, H7), 1.36-1.25 (m, 1H, H2) and 0.95 (t, 3H, J 7.5, H8); δ_{C} (100 MHz, CDCl₃) 175.6 (C═O), 148.3 (C-Bu), 137.1 (C—SO₂), 129.1, 127.0 (CH phenyl), 55.3 (CH—NH), 42.1 (C1), 35.5 (C4), 33.3 (C5), 33.1 (C3), 28.6 (C6), 28.0 (C7), 22.3 (C2) and 13.9 (C8); ESI m/z 100%, 670.6 (M₂Na⁺), 86%, 347.1 (MNa⁺) and 43%, 325.1 (MH⁺).

### Example 15

### (S)-3-(4′-tert-Butylbenzenesulfonylamino)-azepan-2-one

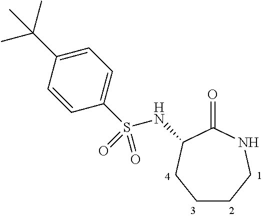 [SMILES: CC(C)(C)c1ccc(S(=O)(=O)N[C@H]2CCCCNC2=O)cc1]

((S)-3-amino-azepan-2-one hydrochloride (2.35 g, 9.18 mmoles) was dissolved in H₂O (20 mL) and cooled to 0° C. 4-^{t}Butylbenzenesulfonyl chloride (1.92 g, 8.25) in THF (40 mL) was added and triethylamine (3.5 mL, 25 mmoles) and the reaction was stirred over night. The THF was removed in vacuo and the product was dissolved in ethyl acetate and washed with pH 2 buffer (3×20 mL) and reduced in vacuo. The product was purified by silica column chromatography (petroleum ether:ethyl acetate 50:50:0 to 0:80:20) to give the product as a white solid 0.67 g (25%); nip 189-190° C.; δ_{H} (400 MHz, CDCl₃) 7.74 (d, 2H, J 8.5, CC—C-^{t}Bu), 7.47 (d, 2H, J 8.5, CH—C—SO₂), 6.27 (br.t, 1H, J 6.5, NH—C1), 6.19 (d, 1H, J4.5, NH—CH), 3.88-3.81 (m, 1H, CH—C4), 3.21-3.12 (m, 1H, H1), 3.05 (ddd, 1H, J 14.5, 11.5, 5, H1), 2.19-2.13 (m, 1H, H4), 2.02-1.96 (m, 1H, H3), 1.81-1.74 (m, 1H, H2), 1.71-1.54 (m, 2H, H3 & H4), 1.33-1.29 (m, 1H, H2) and 1.31 (s, 3H, C(CH₃)₃); δ_{C} (100 MHz, CDCl₃) 175.5 (C═O), 156.4 (C—C(CH₃)₃), 137.0 (C—C═O), 126.8, 126.1 (CH phenyl), 55.4 (CH—NH), 42.3 (C1), 35.2 (C(CH₃)₃), 33.5 (C4), 31.1 (C(CH₃)₃) and 28.7 (C2), 28.0 (C3); νₘₐₓ/cm⁻¹ 3219 (NH indole), 2968 (C—H), 1668 (amide C═O), 1594 (aromatic), 1361 (SO₂) and 1159 (SO₂); ESI m/z 100%, 347.1 (MNa⁺) and 26%, 670.6 (M₂Na⁺); HR ESI m/z (C₁₆H₂₄N₂O₃SH⁺ requires 325.1580) found 325.1580; [α]²⁴_{D} (c =0.532, CHCl₃) 109.68.

### Example 16

### (S)-3-(4′-tert-Butylbenzenesulfonylamino)-tetrahydropyridin-2-one

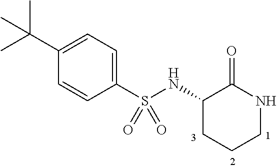 [SMILES: CC(C)(C)c1ccc(S(=O)(=O)N[C@H]2CCCNC2=O)cc1]

(S)-3-amino-γ-lactam (10 mmoles) was dissolved in H₂O (40 mL) and cooled to 0° C. 4-tButylbenzenesulfonyl chloride (1.88 g, 8.08 mmoles) in dichloromethane (25 mL) was added and triethylamine (3.5 mL, 25 mmoles) and the reaction was stirred over night. The reaction was extracted with dichloromethane (3×20 mL), the organic layer was washed with a pH 2 buffer (3×20 mL) and reduced in vacuo. The product was purified by silica column chromatography (petroleum ether:ethyl acetate:methanol 50:50:0 to 0:80:20) to give the product as a white solid 0.76 g (30%); mp 155-156° C.; δ_{H} (400 MHz, CDCl₃) 7.79 (d, 2H, J 8.5, CH—C-^{t}Bu), 7.47 (d, 2H, J 8.5, CH—SO₂), 6.38 (br.s, 1H, NH—C1), 5.98 (d, 1H, J 3.5, NH—CH), 3.51-3.46 (m, 1H, CH—C4), 3.28-3.22 (m, 2H, H1), 2.49-2.42 (m, 1H, H3), 1.93-1.87 (m, 1H, H2), 1.86-1.67 (m, 2H, H2 & H3) and 1.31 (s, 9H, C(CH₃)₃); δ_{C} (100 MHz, CDCl₃) 170.1 (C═O lactam), 156.5 (C—C(CH₃)₃), 136.0 (C—SO₂), 127.1, 126.2 (CH phenyl), 53.2 (CH—NH), 41.8 (Cl), 35.2 (C(CH₃)₃), 31.1 (C(CH₃)₃), 28.4 (C3) and 20.7 (C2); ESI m/z 100%, 333.1 (MNa⁺) and 44%, 642.6 (M₂Na⁺); νₘₐₓ/cm⁻¹: 3220 (NH), 2946 (C—H), 1665, 1596 (aromatic), 1331 (SO₂) and 1134 (SO₂); HR ESI m/z (C₁₅H₂₂N₂O₃SH⁺ requires 311.1424) found 311.1425; [α]²⁴_{D} (c=0.525, CHCl₃)+122.92.

### Example 17

### (R)-3-(4′-tert-Butylbenzenesulfonylamino)-tetrahydropyridin-2-one

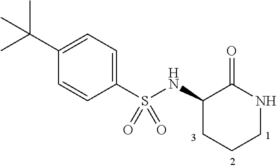 [SMILES: CC(C)(C)c1ccc(S(=O)(=O)N[C@@H]2CCCNC2=O)cc1]

(R)-3-amino-γ-lactam (2.5 mmoles) was dissolved in H₂O (30 mL) and cooled to 0° C. 4-^{t}Butylbenzenesulfonyl chloride (0.61 g, 2.62 mmoles) in dichloromethane (25 mL) was added and triethylamine (1.1 mL, 7.5 mmoles) and the reaction was stirred over night. The reaction was extracted with dichloromethane (3×20 mL), the organic layer was washed with a pH 2 buffer (3×20 mL) and reduced in vacuo. The product was purified by silica column chromatography (petroleum ether:ethyl acetate 75:25 to 0:100) to give the product as a white solid 0.33 g (43%); δ_{H} (400 MHz, CDCl₃) 7.80 (d, 2H, J 8.5, CH—C-^{t}Bu), 7.50 (d, 2H, J 8.5, CH—SO₂), 5.99 (br.s, 1H, NH—C1), 5.90 (br.d, 1H, J 2, NH—CH), 3.51-3.46 (m, 1H, CH—C4), 3.31-3.26 (m, 2H, H1), 2.53-2.43 (m, 1H, H3), 1.96-1.89 (m, 1H, H2), 1.85-1.69 (m, 2H, H2 & H3) and 1.32 (s, 9H, C(CH₃)₃); δ_{C} (100 MHz, CDCl₃) 169.9 (C═O lactam), 156.6 (C—C(CH₃)₃), 135.8 (C—SO₂), 127.2, 126.2 (CH phenyl), 53.3 (CH—NH), 42.0 (C1), 35.2 (C(CH₃)₃), 31.1 (C(C—H₃)₃), 28.5 (C3) and 20.8 (C2); ESI m/z 37%, 642.6 (M₂Na⁺); HR ESI m/z (C₁₅H₂₂N₂O₃SH⁺ requires 311.1424) found 311.1427; [α]²⁴_{D}(c=0.114, CHCl₃) −116.52.

### Example 18

### (S)-3-(4′-Octylbenzenesulfonylamino)-azepan-2-one

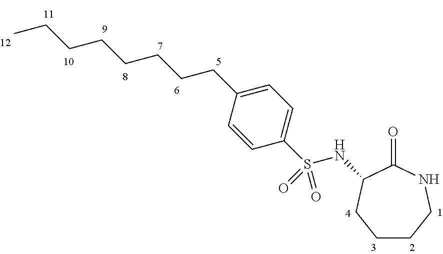 [SMILES: CCCCCCCCc1ccc(S(=O)(=O)N[C@H]2CCCCNC2=O)cc1]

(5)-3-amino-azepan-2-one hydrochloride (0.73 g, 4.45 mmoles) was dissolved in H₂O (30 mL) and cooled to 0° C. 4-Octylbenzenesulfonyl chloride (2.2 mmoles) in dichloromethane (25 mL) was added and triethylamine (0.93 mL, 6.6 mmoles) and the reaction was stirred over night. The reaction was extracted with dichloromethane (3×20 mL) the organic layer was washed with a pH 2 buffer (3×20 mL) and reduced in vacuo. The product was purified by silica column chromatography (petroleum ether:ethyl acetate 50:5 to 0:100) to give the product as a white solid 0.49 g (59%); δ_{H} (400 MHz, CDCl₃) 7.71 (d, 2H, J 8.5, CH—C-Oct), 7.25 (d, 2H, J 8.5, CH—CSO₂), 6.68 (dd, 1H, J 7.5, 5,5 NH—C1), 6.22 (d, 1H, J 5, NH—CH), 3.81 (ddd, 1H, J 2, 5, 11 CH—C4), 3.20-3.311 (m, 1H, H1), 3.02 (ddd, 1H, J 16, 11.5, 5, H1), 2.63 (t, 2H, J 8, H5), 2.12-2.07 (m, 1H, H4), 1.98-1.93 (m, 1H, H3), 1.79-1.72 (m, 1H, H2), 1.65-1.55 (m, 4H, H3, H4 & H5), 1.33-1.21 (m, 11H, H2, H7, H8, H9, H10 & H11) and 0.86 (t, 3H, J 7, H12); δ_{C} (100 MHz, CDCl₃) 174.6 (C═O lactam), 148.4 (C—Oct), 137.2 (C—SO₂), 129.1, 128.8 (CH phenyl), 127.0 (CH phenyl), 55.3 (CH—NH), 42.1 (C1), 35.8 (C4), 33.3 (C5), 31.8 (C3), 31.0 (C6), 29.4 (C7), 29.2 (C8), 28.6 (C9), 28.0 (C10), 22.7 (C2), 22.6 (C11) and 14.1 (C12); ESI m/z 100%, 403.2 (MNa⁺) and 40%, 381.2 (MH⁺); HR ESI m/z (C₂₀H₃₂N₂O₃SH⁺ requires 381.2206) found 381.2205.

### Example 19

### (S)-3-(4′-Octylbenzenesulfonylamino)-tetrahydropyridin-2-one

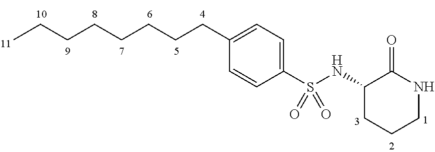 [SMILES: CCCCCCCCc1ccc(S(=O)(=O)N[C@H]2CCCNC2=O)cc1]

(S)-3-amino-γ-lactam (2.5 mmoles) was dissolved in H₂O (40 mL) and cooled to 0° C. 4-Octylbenzenesulfonyl chloride (1.34 mmoles) in dichloromethane (25 mL) was added and triethylamine (0.57 mL, 4 mmoles) and the reaction was stirred over night. The reaction was extracted with dichloromethane (3×20 mL) the organic layer was washed with a pH 2 buffer (3×20 mL) and reduced in vacuo. The product was purified by silica column chromatography (petroleum ether:ethyl acetate:methanol 50:50:0 to 0:80:20) to give the product as a white solid 0.31 g (63%); mp 98-99° C.; δ_{H} (400 MHz, CDCl₃) 7.76 (d, 2H, J, CH—C-Oct), 7.27 (d, 2H, J 8, CH—CSO₂), 6.50 (br,s, 1H, NH—C1), 6.00 (d, 1H, J 2.5, NH—CH), 3.51-3.45 (m, 1H, CH—C3), 3.27-3.31 (m, 2H, H1), 2.62 (t, 2H, J 7, H4), 2.45-2.36 (m, 1H, H3), 1.92-1.85 (m, 1H, H2), 1.80-1.67 (m, 2H, H2 & H3), 1.62-1.55 (m, 2H, H5), 1.31-1.20 (m, 10H, H6, H7, H8, H9 & H10) and 0.85 (t, 3H, J 7, H11); δ_{C} (100 MHz, CDCl₃) 170.1 (C═O lactam), 148.5 (C—Oct), 136.3 (C—SO₂), 129.1 (CH phenyl), 127.3 (CH phenyl), 53.2 (CH—NH), 41.8 (C1), 35.9 (C4), 31.8 (C5), 31.0 (C3), 29.4 (C6), 29.3 (C7), 29.2 (C8), 28.4 (C9), 22.7 (C10), 20.7 (C2) and 14.1 (C11); ESI m/z 100%, 389.2 (MNa⁺) and 36%, 367.2 (MH⁺); HR ESI m/z (C₁₉H₃₀N₂O₃SNa⁺ requires 389.1869) found 389.1865; νₘₐₓ/cm⁻¹ : 3207 (NH), 2920 (C—H), 1664, (C═O), 1544 (aromatic), 1310 (SO₂) and 1188 (SO₂); [α]²⁴_{D} (c=0.515, CHCl₃) +99.97.

### Example 20

### (R)-3-(4′-Octylbenzenesulfonylamino)-tetrahydropyridin-2-one

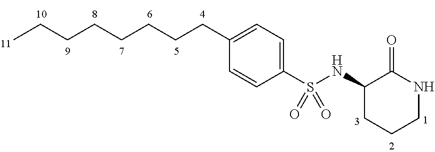 [SMILES: CCCCCCCCc1ccc(S(=O)(=O)N[C@@H]2CCCNC2=O)cc1]

(R)-3-amino-γ-lactam (2.5 mmoles) was dissolved in H₂O (30 mL) and cooled to 0° C. 4-Octylbenzenesulfonyl chloride (1.34 mmoles) in dichloromethane (25 mL) was added and triethylamine (0.57 mL, 4 mmoles) and the reaction was stirred over night. The reaction was extracted with dichloromethane (3×20 mL) the organic layer was washed with a pH 2 buffer (3×20 mL) and reduced in vacuo. The product was purified by silica column chromatography (petroleum ether:ethyl acetate 50:50 to 0:100) to give the product as a white solid 0.22 g (45%); δ_{H} (400 MHz, CDCl₃) 7.76 (d, 2H, J 8, CH—C-Oct), 7.26 (d, 2H, J 8, CC—CSO₂), 6.85 (br.s, 1H, NH—C1), 6.14 (d, 1H, J 3, NH—CH), 3.52-3.46 (m, 1H, CH—C3), 3.23-3.16 (m, 2H, H1), 2.62 (t, 2H, J 7, H4), 2.38-2.31 (m, 1H, H3), 1.87-1.81 (m, 1H, H2), 1.74-1.65 (m, 2H, H2 & H3), 1.62-1.54 (m, 2H, H5), 1.32-1.20 (m, 10H, H6, H7, H8, H9 & H10) and 0.84 (t, 3H, J 7, H11); δ_{C} (100 MHz, CDCl₃) 170.2 (C═O lactam), 148.4 (C-Oct), 136.5 (C—SO₂), 129.0 (CH phenyl), 127.3 (CH phenyl), 53.2 (C—NH), 41.5 (C1), 35.8 (C4), 32.0 (C5), 31.8 (C3), 29.4 (C6), 29.3 (C7), 29.1 (C8), 25.5 (C9), 22.7 (C10), 20.7 (C2) and 14.1 (C11); ESI m/z 15%, 389.2 (MNa⁺); HR ESI m/z (C₁₉H₃₀N₂O₃SNa⁺ requires 389.1869) found 389.1872; [α]²⁴_{D}(c=0.238, CHCl₃) −102.94.

### Pharmacological Study of the Products of the Invention

A. Inhibition of MCP-1 Induced Leukocyte Migration

Assay Principle

The biological activity of the compounds of the current invention may be demonstrated using any of a broad range of functional assays of leukocyte migration in vitro, including but not limited to Boyden chamber and related transwell migration assays, under-agarose migration assays and direct visualisation chambers such as the Dunn Chamber.

For example, to demonstrate the inhibition of leukocyte migration in response to chemokines (but not other chemoattractants) the 96-well format micro transwell assay system from Neuroprobe (Gaithersburg, Md., USA) has been used. In principle, this assay consists of two chambers separated by a porous membrane. The chemoattractant is placed in the lower compartment and the cells are placed in the upper compartment. After incubation for a period at 37° C. the cells move towards the chemoattractant, and the number of cells in the lower compartment is proportional to the chemoattractant activity (relative to a series of controls).

This assay can be used with a range of different leukocyte populations. For example, freshly prepared human peripheral blood leukocytes may used. Alternatively, leukocyte subsets may be prepared, including polymorphonuclear cells or lymphocytes or monocytes using methods well known to those skilled in the art such as density gradient centrifugation or magnetic bead separations. Alternatively, immortal cell lines which have been extensively validated as models of human peripheral blood leukocytes may be used, including, but not limited to THP-1 cells as a model of monocytes or Jurkat cells as model of naive T cells.

Although a range of conditions for the assay are acceptible to demonstrate the inhibition of chemokine-induced leukocyte migration, a specific example is hereby provided.

Materials

The transwell migration systems are manufactured by Neuroprobe, Gaithersburg, Md., USA.

The plates used are ChemoTx plates (Neuroprobe 101-8) and 30 μl clear plates (Neuroprobe MP30).

Geys' Balanced Salt Solution is purchased from Sigma (Sigma G-9779).

Fatty acid-free BSA is purchased from Sigma (Sigma A-8806).

MTT, i.e. 3-(4,5-dimethylthiazol-2-yl)-2,5-diphenyltetrazolium bromide, is purchased from Sigma (Sigma M-5655).

RPMI-1640 without phenol red is purchased from Sigma (Sigma R-8755).

The THP-1 cell line (European Cell culture Collection) were used as the leukocyte cell population.

Test Protocol

The following procedure is used for testing the invention compounds for MCP-1 induced leukocyte migration:

First, the cell suspension to be placed in the upper compartment is prepared. The THP-1 cells are pelleted by centrifugation (770×g; 4 mins) and washed with Geys Balanced Salt Solution with 1 mg/ml BSA (GBSS+BSA). This wash is then repeated, and the cells repelleted before being resuspended in a small volume of GBSS+BSA for counting, for example using a standard haemocytometer.

The volume of GBSS+BSA is then adjusted depending on the number of cells present so that the cells are at final density of 4.45×10⁶ cells per ml of GBSS +BSA. This ensures that there are 100,000 THP-1 cells in each 25 μl of the solution that will be placed in the upper chamber of the plate.

To test a single compound for its ability to inhibit MCP-1 induced migration, it is necessary to prepare two lots of cells. The suspension of THP-1 cells at 4.45×10⁶ cells/ml is divided into two pots. To one pot the inhibitor under test is added at an appropriate final concentration, in an appropriate vehicle (for example at 1 μM in not more than 1% DMSO). To the second pot an equal volume of GBSS+BSA plus vehicle as appropriate (e.g. not more than 1% DMSO) is added to act as a control.

Next, the chemoattractant solution to be placed in the lower compartment is prepared. MCP-1 is diluted in GBSS+BSA to give a final concentration of 25 ng/ml. This is divided into two pots, as for the cell suspension. To one pot, the test compound is added to the same final concentration as was added to the cell suspension, while to the other pot an equal volume of GBSS+BSA plus vehicle as appropriate (e,g. not more than 1% DMSO) is added.

Note that the volume of liquid that needs to be added to make the addition of the text compound needs to be taken into account, when establishing the final concentration of MCP-1 in the solution for the lower compartment and the final concentration of cells in the upper compartment.

Once the chemoattractant solutions for the lower wells and cell solutions for the upper chambers have been prepared, the migration chamber should be assembled. Place 29 μl of the appropriate chemoattractant solution into the lower well of the chamber. Assays should be performed with at least triplicate determinations of each condition. Once all the lower chambers have been filled, apply the prous membrane to the chamber in accordance with the manufacturer's instructions. Finally, apply 25 μl of the appropriate cell solution to each upper chamber. A plastic lid is placed over the entire apparatus to prevent evaporation.

The assembled chamber is incubated at 37° C., 5% CO₂, for 2 hours. A suspension of cells in GBSS+BSA is also incubated under identical conditions in a tube: these cells will be used to construct a standard curve for determining the number of cells that have migrated to the lower chamber under each condition.

At the end of the incubation, the liquid cell suspension is gently removed from the upper chamber, and 20 μl of ice-cold 20 mM EDTA in PBS is added to the upper chamber, and the apparatus is incubated at 4° C. for 15 mins. This procedure causes any cells adhering to the underside of the membrane to fall into the lower chamber.

After this incubation the filter is carefully flushed with GBSS+BSA to wash off the EDTA, and then the filter is removed.

The number of cells migrated into the lower chamber under each condition can then be determined by a number of methods, including direct counting, labelling with fluorescent or radioactive markers or through the use of a vital dye. Typically, we utilise the vital dye MTT. 3 μl of stock MTT solution are added to each well, and then the plate is incubated at 37° C. for 1-2 hours during which time dehydrogenase enzymes within the cells convert the soluble MTT to an insoluble blue formazan product that can be quantified spectrophotometrically.

In parallel, an 8-point standard curve is set up. Starting with the number of cells added to each upper chamber (100,000) and going down in 2-fold serial dilutions in GBSS +BSA, the cells are added to a plate in 25 μl, with 3 μl of MU stock solution added. The standard curve plate is incubated along side the migration plate.

At the end of this incubation, the liquid is carefully removed from the lower chambers, taking care not to disturb the precipitated formazan product. After allowing to air dry briefly, 20 μl of DMSO is added to each lower chamber to solubilise the blue dye, and absorbance at 595 nm is determined using a 96-well plate reader. The absorbance of each well is then interpolated to the standard curve to estimate the number of cells in each lower chamber.

The MCP-1 stimulated migration is determined by subtracting the average number of cells that reached the lower compartment in wells where no MCP-1 was added from the average number of cells that reached the lower compartment where MCP-1 was present at 25 ng/ml.

The impact of the test substance is calculated by comparing the MCP-1-induced migration which occurred in the presence or absence of various concentrations of the test substance. Typically, the inhibition of migration is expressed as a percentage of the total MCP-1 induced migration which was blocked by the presence of the compound. For most compounds, a dose-response graph is constructed by determining the inhibition of MCP-1 induced migration which occurs at a range of different compound concentrations (typically ranging from 1 nM to 1 μM or higher in the case of poorly active compounds). The inhibitory activity of each compound is then expressed as the concentration of compound required to reduce the MCP-1-induced migration by 50% (the ED₅₀ concentration).

Results

The compounds of reference examples 1 to 2 were tested and were shown to have an ED₅₀ of 100 nM or less in this test.

B. In Vivo Assay

The anti-inflammatory efficacy of an exemplar compound according to the present invention was tested using the murine sub-lethal endotoxemia model. This model has been widely used to demonstrate the anti-inflammatory effect of compounds in vivo—see for example Fox et al., 2009, J Med Chem. 52(11): 3591-3595.

Briefly, the method is as follows. Female CD1 mice (28-30g, ˜7 weeks of age) were dosed with their respective treatment in sterile filtered 1% CMC by oral gavage in a dose volume of 10 ml/kg one hour prior to an endotoxin (LPS) challenge. The endotoxin challenge was injected by the intraperitoneal route containing 675,000 Endotoxin Units of LPS (E. coli strain 0111:B4 (Code L4130)) in endotoxin free PBS. Mice were left for two hours and then exsanguinated under terminal anaesthesia and blood was taken. Serum was prepared from this terminal bleed and aliquoted and stored at −20° C. Serum TNF-α levels were measured by ELISA per manufacturers instructions (R and D Systems).

Eight animals were treated in each group, and the data for the animal with the highest and lowest TNF-α level in each group were eliminated, and the mean and standard error reported for the remaining six animals.

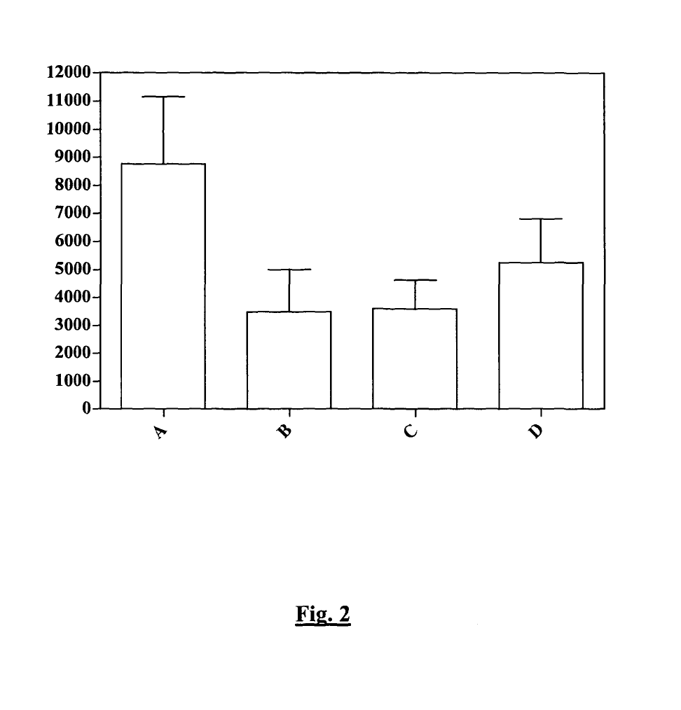

A single dose of (S)-3-(4′-trifluoromethylbenzenesulfonylamino)-tetrahydropyridin-2-one (see Example 4; compound also known as (S)—N-(2-Oxopiperidin-3-yl)-4-(trifluoromethyl)-benzenesulfonamide) administered by oral gavage, inhibited endotoxin-stimulated TNF-alpha levels by 60-70% (see FIG. 2). The anti-inflamatory effect on TNF-α levels in vivo using (S)-3-(4′-trifluoromethylbenzenesulfonylamino)-tetrahydropyridin-2-one was quantitatively similar to a maximally effective dose of the positive control compound thalidomide. Thalidomide is used clinically as an anti-inflammatory agent, reducing the level of TNF-α, for the treatment of leprosy. The anti-inflammatory effect in vivo of (S)-3-(4′-trifluoromethylbenzenesulfonylamino)-tetrahydropyridin-2-one was also similar to the earlier somatotaxin (S)-3-(adamantane-1-carbonyl)aminocaprolactam (see WO20061016152).

These data demonstrate that the compounds of the present invention have clinically useful anti-inflammatory activity in vivo, comparable to other anti-inflammatory agents used for the treatment of a wide range of disorders with an inflammatory component.

1. A compound of formula (I), or a pharmaceutically acceptable salt thereof: 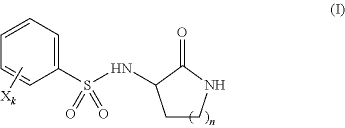
wherein:
n is an integer from 1 to 4;
k is an integer from 0 to 5, representing the number of groups substituting C₂, C₃, C₄, C₅ and/or C₆ of the phenyl ring; and
X are linear or branched groups substituting the phenyl ring independently selected from any one of the groups consisting of: alkyl, haloalkyl, hydroxyalkyl, hydroxy, alkoxy, amino, aminoalkyl, aminodialkyl, carboxyl, and halogen.

2. A compound of formula (I′), or a pharmaceutically acceptable salt thereof: 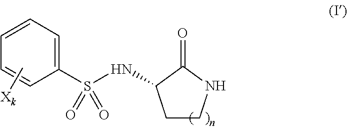
wherein:
n is an integer from 1 to 4;
k is an integer from 0 to 5, representing the number of groups substituting C₂, C₃, C₄, C₅ and/or C₆ of the phenyl ring; and
X are linear or branched groups substituting the phenyl ring independently selected from any one of the groups consisting of: alkyl, haloalkyl, hydroxyalkyl, hydroxy, alkoxy, amino, aminoalkyl, aminodialkyl, carboxyl, and halogen.

3. (canceled)

4. (canceled)

5. A compound according to claim 1, with the proviso that:
when n =3, then at least one of C₂-C₆ on the phenyl ring is substituted with a group other than halogen, C₁-C₇ alkyl, or C₁-C₇ haloalkyl; and
when n =1, 2 or 3, then
C₂ or C₆ on the phenyl ring are other than hydrogen or fluorine, or
C₃ on the phenyl ring is other than hydrogen, halogen, C₁-C₆ alkyl,
C₁-C₆ alkoxy, or C₁-C₆ haloalkyl, or
C₄ on the phenyl ring is other than hydrogen, halogen, C₁-C₆ alkyl, C₁-C₆ alkoxy, C₁-C₆ haloalkyl, amino, aminoalkyl or aminodialkyl, or
C₅ on the phenyl ring is other than hydrogen or halogen;
provided that the compound is neither of:
3-(2′-carboxybenzenesulfonylamino)-tetrahydropyridin-2-one, and (R)-3-(4′-methylbenzenesulfonylamino)-caprolactam.

6. A compound of claim 2, with the proviso that:
when n=3, then at least one of C₂-C₆ on the phenyl ring is substituted with a group other than halogen, C₁-C₇ alkyl, or C₁-C₇ haloalkyl; and
when n =1, 2 or 3, then
C₂ or C₆ on the phenyl ring are other than hydrogen or fluorine, or
C₃ on the phenyl ring is other than hydrogen, halogen, C₁-C₆ alkyl, C₁-C₆ alkoxy, or C₁-C₆ haloalkyl, or
C₄ on the phenyl ring is other than hydrogen, halogen, C₁-C₆ alkyl, C₁-C₆ alkoxy, C₁-C₆ haloalkyl, amino, aminoalkyl or aminodialkyl, or
C₅ on the phenyl ring is other than hydrogen or halogen;
provided that the compound is not one of the group consisting of:
(S)-3-(4′-methylbenzenesulfonylamino)-tetrahydropyridin-2-one,
(S)-3-(4′-methylbenzenesulfonylamino)-caprolactam,
(S)-3-(4′-bromobenzenesulfonylamino)-caprolactam, and
(S)-3-(4′-chlorobenzenesulfonylamino)-caprolactam.

7. A pharmaceutical composition comprising, as active ingredient, a compound as defined in claim 5, or a pharmaceutically acceptable salt thereof, and at least one pharmaceutically acceptable excipient and/or carrier.

8. The compound according to claim 1, wherein n=2.

9. The compound according to claim 1, wherein n=3.

10. The compound according to claim 1, wherein X is haloalkyl.

11. A compound according to claim 2, selected from the group consisting of:
(S)-3-(3′-fluorobenzenesulfonylamino)-tetrahydropyridin-2-one,
(S)-3-(4′-fluorobenzenesulfonylamino)-tetrahydropyridin-2-one,
(S)-3-(2′-trifluoromethylbenzenesulfonylamino)-tetrahydropyridin-2-one,
(S)-3-(3′-trifluoromethylbenzenesulfonylamino)-tetrahydropyridin-2-one,
(S)-3-(4′-trifluoromethylbenzenesulfonylamino)-tetrahydropyridin-2-one,
(S)-3-(2′,4′-difluorobenzenesulfonylamino)-tetrahydropyridin-2-one,
(S)-3-(2′,5-difluorobenzenesulfonylamino)-tetrahydropyridin-2-one,
(S)-3-(2′,6′-difluorobenzenesulfonylamino)-tetrahydropyridin-2-one,
(S)-3-(3′,4′-difluorobenzenesulfonylamino)-tetrahydropyridin-2-one,
(S)-3-(3′,5-difluorobenzenesulfonylamino)-tetrahydropyridin-2-one,
(S)-2-fluoro-N-(2-oxopiperidin-3-yl)benzenesulfonamide,
(S)-3-(4′-ethylbenzenesulfonylamino)-azepan-2-one,
(S)-3-(4′-butylbenzenesulfonylamino)-azepan-2-one,
(S)-3-(4′-tert-butylbenzenesulfonylamino)-azepan-2-one,
(S)-3-(4′-tert-butylbenzenesulfonylamino)-tetrahydropyridin-2-one,
(S)-3-(4′-octylbenzenesulfonylamino)-azepan-2-one,
(S)-3-(4′-methylbenzenesulfonylamino)-caprolactam, and
(S)-3-(4′-octylbenzenesulfonylamino)-tetrahydropyridin-2-one,
and pharmaceutically acceptable salts thereof.

12. A compound according to claim 1, selected from the group consisting of:
(R)-3-(4′-ethylbenzenesulfonylamino)-tetrahydropyridin-2-one,
(R)-3-(4′-tert-Butylbenzenesulfonylamino)-tetrahydropyridin-2-one, and
(R)-3-(4′-octylbenzenesulfonylamino)-tetrahydropyridin-2-one, and pharmaceutically acceptable salts thereof.

13. (canceled)

14. The compound of claim 11 having formula (S)-3-(4′-methylbenzenesulfonylamino)-caprolactam, or a pharmaceutically acceptable salt thereof.

15. The compound of claim 11 having formula (S)-3-(4′-trifluoromethylbenzenesulfonylamino)-tetrahydropyridin-2-one, or a pharmaceutically acceptable salt thereof.

16. A method of treating an inflammatory disorder, the method comprising:
administering to a subject in need thereof, a compound according to claim 1,
wherein the inflammatory disorder is selected from the group consisting of:
autoimmune diseases, asthma, rheumatoid arthritis, a disorder characterised
by an elevated TNF-α level, psoriasis, allergies, multiple sclerosis, fibrosis, diabetic nephropathy, and formation of adhesions.

17. The method according to claim 16, wherein the inflammatory disorder is formation of adhesions.

18. The method according to claim 17, wherein the compound is administered locally.

19. (canceled)

20. (canceled)

21. A pharmaceutical composition comprising, as active ingredient, a compound as defined in claim 6, or a pharmaceutically acceptable salt thereof, and at least one pharmaceutically acceptable excipient and/or carrier.

22. The compound according to claim 2, wherein n=2.

23. The compound according to claim 2, wherein n=3.

24. The compound according to claim 2, wherein X is haloalkyl.

25. A method of treating an inflammatory disorder, the method comprising:
administering to a subject in need thereof, a compound according to claim 2, wherein the inflammatory disorder is selected from the group consisting of:
autoimmune diseases, asthma, rheumatoid arthritis, a disorder characterised by an elevated TNF-α level, psoriasis, allergies, multiple sclerosis, fibrosis, diabetic nephropathy, and formation of adhesions.
